(19) United States
(12) Patent Application Publication
(10) Pub. No.: US 2021/0117933 A1
(43) Pub. Date: Apr. 22, 2021

(54) PERSONAL PROTECTIVE EQUIPMENT MANAGEMENT SYSTEM WITH DISTRIBUTED DIGITAL BLOCKCHAIN LEDGER

(71) Applicant: 3M INNOVATIVE PROPERTIES COMPANY, St. Paul, MN (US)

(72) Inventors: Eric C. Lobner, Woodbury, MN (US); Oscar G. Naim D'Paola, Lake Elmo, MN (US); Michael L. Gannon, Mendota Heights, MN (US); Joseph P. Kronzer, Shoreview, MN (US); Johannes P.M. Kusters, Austin, TX (US); Lyle L. Luppes, Rosemount, MN (US)

(21) Appl. No.: 17/250,013

(22) PCT Filed: Apr. 17, 2019
(86) PCT No.: PCT/IB2019/053191

§ 371 Date: Nov. 2, 2020

Related U.S. Application Data
(60) Provisional application No. 62/666,602, filed on May 3, 2018.

(51) Int. Cl. G06Q 10/00; G06Q 10/08; G06Q 10/06; G16H 40/67; G06Q 30/00; G06Q 10/10; G06F 16/27; G06F 16/23

(52) U.S. Cl. CPC G06Q 10/20; G06Q 10/087; G06Q 10/06395; G16H 40/67; A61B 5/6803; G06Q 10/105; G06F 16/27; G06F 16/2379; G06Q 30/0185

(57) ABSTRACT

A system includes a computing device and an article of personal protective equipment (PPE). The article of PPE includes at least one sensor configured to generate a stream of PPE data. The computing device is configured to store the PPE data in transaction data that is stored by a distributed ledger managed by a consensus network of computing devices. The computing device is further configured to perform at least one action based at least in part on the transaction data stored by the distributed ledger.

### TECHNICAL FIELD

The present disclosure relates generally to articles of personal protective equipment and computing systems related to use of personnel protective equipment.

### BACKGROUND

Personal protective equipment (PPE) may be used to help protect a user (e.g., a worker) from harm or injury from a variety of causes. For example, workers may wear eye protection, such as safety glasses, in many different work environments. As another example, workers may use fall protection equipment when operating at potentially harmful or even deadly heights. As yet another example, when working in areas where there is known to be, or there is a potential of there being, dusts, fumes, gases or other contaminants that are potentially hazardous or harmful to health, it is usual for a worker to use a respirator or a clean air supply source, such as a powered air purifying respirators (PAPR) or a self-contained breathing apparatus (SCBA). Other PPE may include, as non-limiting examples, hearing protection, head protection (e.g., visors, hard hats, or the like), protective clothing, or the like.

### SUMMARY

In general, the present disclosure describes techniques in which a personnel protective equipment (PPE) management system implements one or more distributed digital ledgers using blockchains and peer-to-peer networks to provide enhanced PPE authentication, tracking and management as well as safety event monitoring and compliance reporting. In some examples, articles of PPE may be enhanced to generate PPE data including usage data, maintenance data, inspection data, or a combination therein and may register the data with the distributed ledger. As one example, a variety of PPEs and/or other components of a work environment may be fitted with electronic sensors that generate streams of data regarding status or operation of the PPE, environmental conditions within regions of the work environment, and the like. The PPE management system presents an interface for receiving the streams of data and is configured to register the data within the secure, distributed ledger maintained by the peer-to-peer network. Registering usage data, maintenance data, and/or inspection data with the distributed ledger may increase security of the data. For example, because distributed ledgers are immutable, the techniques of this disclosure may enable PPE data to be stored more accurately and securely, which may reduce risk of counterfeit articles of PPE or fraudulent data, thereby increasing worker safety. In some examples, data in the distributed ledgers may be used by one or more computing devices to detect safety events with higher levels of confidence because the data may be immutable and/or available in a distributed architecture that is available in a variety of different physical settings.

As one example, counterfeit, fake, or otherwise untested and/or approved personnel protective equipment may intentionally or inadvertently be used in safety-controlled work environments. These ‘non-authenticated’ PPEs are often inferior to the PPEs that have been approved for the work environment and for which the individual work has likely been fit tested and trained. Intentional or inadvertent use of non-authenticated PPEs instead of authenticated PPEs may increase risk to workers utilizing the personnel protective equipment.

The articles, systems and techniques described herein provide certain technical advantages for improved authentication of articles of personnel protective equipment (PPE) as well as the data generated by PPEs. As one example, a manufacturer of PPE may mark articles of PPE with a code and may register the code with the PPE management system that utilizes a secure, distributed ledger (e.g., blockchain) managed by a consensus network of peer-to-peer computing nodes. As one example, a code may be a Quick Response (QR) or other machine-readable code, for instance. A computing system used by a worker utilizing an article of PPE may scan the code and verify the authenticity of the article of PPE based on the distributed ledger maintained by the PPE management systems.

In this way, the PPE management system provides a centralized interface for maintaining one or more underlying distributed ledgers for workplaces and/or enterprises to include the data generated by the PPEs and/or environmental sensors, such as thermometers or gas detectors, that are located in the work environment(s). For example, the environmental sensors may monitor corresponding environmental conditions of the environment and provide environmental data indicating the environmental conditions to the distributed ledger managed by the consensus network. The distributed ledger managed by the consensus network may also include equipment data, hazard data, worker data or a combination therein. Storing such data in a distributed ledger managed by a consensus network may increase the security of such data by preventing fraudulent data entries and providing redundant data across multiple computing devices.

In one example, the disclosure describes a system that includes an article of personal protective equipment (PPE) and at least one computing device. The article of PPE includes at least one sensor configured to generate a stream of PPE data. The at least one computing device is configured to store, in transaction data stored by a distributed ledger managed by a consensus network of computing devices, the PPE data, and perform, based at least in part on the transaction data stored by the distributed ledger, at least one action.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a block diagram illustrating an example system in which a personnel protective equipment (PPE) management system implements one or more distributed digital ledgers using blockchains and peer-to-peer networks to provide enhanced PPE authentication, tracking and management as well as safety event monitoring and compliance reporting, in accordance with various techniques of this disclosure.

FIG. 2 is a block diagram illustrating, in detail, an operating perspective of the PPE management system shown in FIG. 1 when hosted as a cloud-based platform capable of supporting multiple, distinct work environments having an overall population of workers, in accordance with various techniques of this disclosure.

FIG. 3 is a block diagram depicting a system for authenticating an article of personal protective equipment, according to techniques described in this disclosure.

FIG. 4 is a flow diagram illustrating example operations of a computing device configured to authenticate an article of personal protective equipment, in accordance with one or more techniques of this disclosure.

FIG. 5 is flowchart illustrating an example mode of operation for a consensus network, according to techniques of this disclosure.

FIG. 6 is flowchart illustrating an example mode of operation for a consensus network, according to techniques of this disclosure.

FIG. 7 is flowchart illustrating an example mode of operation for a consensus network, according to techniques of this disclosure.

FIG. 8 is flowchart illustrating an example mode of operation for a consensus network, according to techniques of this disclosure.

It is to be understood that the embodiments may be utilized and structural changes may be made without departing from the scope of the invention. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

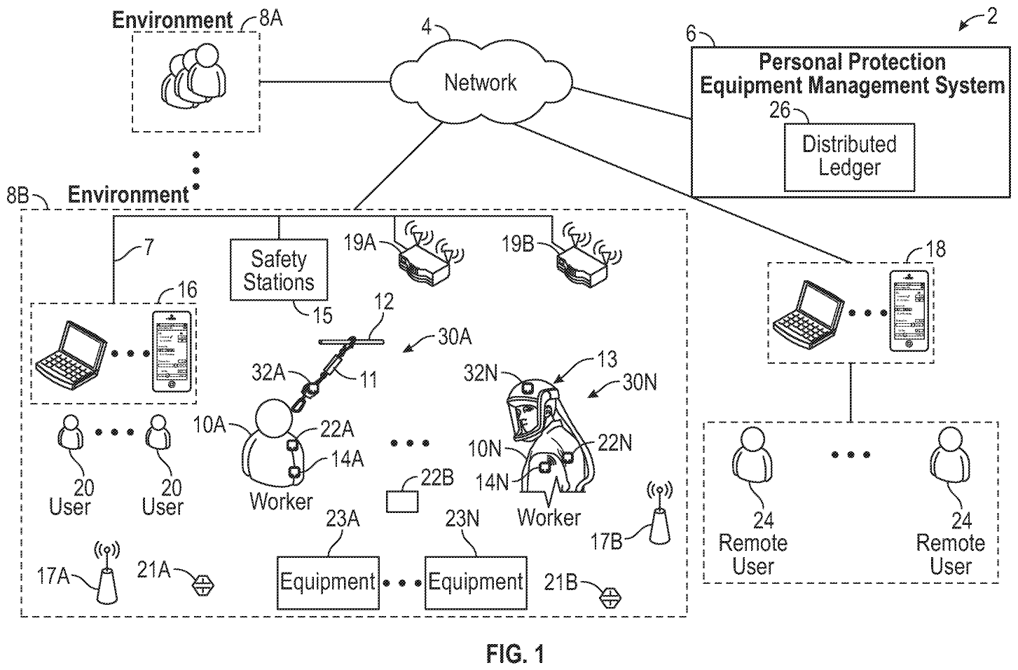

FIG. 1 is a block diagram illustrating an example computing system 2 that includes a personal protection equipment management system (PPEMS) 6 implements one or more distributed digital ledgers using blockchains and peer-to-peer networks of consensus nodes to provide enhanced PPE authentication, tracking and management as well as safety event monitoring and compliance reporting. As described herein, PPEMS 6 allows authorized users to perform preventive occupational health and safety actions and manage inspections and maintenance of safety protective equipment. By interacting with PPEMS 6, safety professionals can, for example, manage area inspections, worker inspections, worker health and safety compliance training.

In general, PPEMS 6 provides data acquisition, monitoring, activity logging, reporting, predictive analytics and alert generation. For example, PPEMS 6 includes an underlying analytics and safety event prediction engine and alerting system in accordance with various examples described herein. As further described below, PPEMS 6 provides an integrated suite of personal safety protection equipment management tools and implements various techniques of this disclosure. That is, PPEMS 6 provides an integrated, end-to-end system for managing personal protection equipment, e.g., safety equipment, used by workers 10 within one or more physical environments 8, which may be construction sites, mining or manufacturing sites or any physical environment. The techniques of this disclosure may be realized within various parts of computing environment 2. Although certain examples of this disclosure are provided with respect to certain types of PPE for illustration purposes, the systems, techniques, and devices of this disclosure are applicable to any type of PPE 30.

As shown in the example of FIG. 1, system 2 represents a computing environment in which a computing device within of a plurality of physical environments 8A, 8B (collectively, environments 8) electronically communicate with PPEMS 6 via one or more computer networks 4. Each of physical environment 8 represents a physical environment, such as a work environment, in which one or more individuals, such as workers 10, utilize personal protection equipment while engaging in tasks or activities within the respective environment.

In this example, environment 8A is shown as generally as having workers 10, while environment 8B is shown in expanded form to provide a more detailed example. In the example of FIG. 1, a plurality of workers 10A-10N are shown as utilizing PPE 30, such as fall protection equipment (shown in this example as self-retracting lifelines (SRLs) 11) attached to safety support structure 12 and respirators 13. As described in greater detail herein, in other examples, workers 10 may utilize a variety of other PPE 30 that is compatible with the techniques described herein, such as hearing protection, head protection, safety clothing, or the like.

As further described herein, each of SRLs 11 includes embedded sensors or monitoring devices and processing electronics configured to capture data in real-time as a user (e.g., worker) engages in activities while wearing the fall protection equipment. In some examples, smart hooks that determine whether a hook is secured or unsecured to a fixed anchoring point may also be within the spirit and scope of fall protection PPE in this disclosure. For example, SRLs may include a variety of electronic sensors such as one or more of an extension sensor, a tension sensor, an accelerometer, a location sensor, an altimeter, one or more environment sensors, and/or other sensors for measuring operations of SRLs 11. In addition, each of SRLs 11 may include one or more output devices for outputting data that is indicative of operation of SRLs 11 and/or generating and outputting communications to the respective worker 10. For example, SRLs 11 may include one or more devices to generate audible feedback (e.g., one or more speakers), visual feedback (e.g., one or more displays, light emitting diodes (LEDs) or the like), or tactile feedback (e.g., a device that vibrates or provides other haptic feedback). Further example details of PPEs that include embedded sensors are described in U.S. provisional patent application 62/408,442, filed Oct. 14, 2016, the entire content of which is hereby expressly incorporated by reference herein.

Respirators 13 may also include embedded sensors or monitoring devices and processing electronics configured to capture data in real-time as a user (e.g., worker) engages in activities while wearing the respirators. For example, as described in greater detail herein, respirators 13 may include a number of components (e.g., a head top, a blower, a filter, and the like) and may include a number of sensors for sensing or controlling the operation of such components. A head top may include, as examples, a head top visor position sensor, a head top temperature sensor, a head top motion sensor, a head top impact detection sensor, a head top position sensor, a head top battery level sensor, a head top head detection sensor, an ambient noise sensor, or the like. A blower may include, as examples, a blower state sensor, a blower pressure sensor, a blower run time sensor, a blower temperature sensor, a blower battery sensor, a blower motion sensor, a blower impact detection sensor, a blower position sensor, or the like. A filter may include, as examples, a filter presence sensor, a filter type sensor, or the like. Each of the above-noted sensors may generate usage data.

In some examples, PPE usage data includes data generated by sensors of the PPE, as described above. For example, PPE usage data generated by sensors of the PPE may include worker temperature, PPE temperature, position of a PPE component (e.g., open, closed, etc.), motion of the PPE, remaining useful life (e.g., remaining battery or filter), among others. PPE usage data may include data indicative of PPE performance or operational status, such as current settings, uptime or runtime, data indicating start and/or stop times, among others.

While FIG. 1 is described with respect to SRLs 11 and respirators 13, as described herein, the techniques of this disclosure may also be applied to a variety of other PPE 30. For example, PPE 30 may include hearing protection, vision protection, and head protection, as well as protective clothing, trauma protection, other PPE for assisted/protective respiration, and so forth. In some examples, PPE 30 includes computerized devices, such as a watch (e.g., a smartwatch), fitness tracker, eyewear, headphones, mobile phone, heart rate monitor, pulse oximeter, or other wearable device that include one or more sensors to monitor workers 10.

PPE 30A-30N may include one or more codes 32A-32N (“codes 32”). In some examples, code 32 may include authentication data that is printed, formed, or otherwise embodied on PPE 30 that facilitates the detection of counterfeit articles. Codes 32 includes authentication data that is machine readable. In some examples, the authentication data of codes 32 corresponds to data stored by a distributed ledger (e.g., blockchain) managed by a consensus network. In some instances, the authentication data includes a unique identifier (e.g., a serial number) corresponding to the respective article of PPE 30. The authentication data may include an indication of a type of article of PPE 30 (e.g., a general type such as respirator or SRL, a more specific type such as air tank or breathing mask, a manufacturer or model number, etc.). In some examples, codes 32 may each represent a string of characters, a number, a QR code, a bar code, or other optical pattern that may encode authentication data (e.g., including unique identifiers).

In some examples, codes 32 may be printed on a label, tape, sticker, or other adhesive device. The adhesive device may include at least one adhesive or cling-film layer to affix to PPE 30. In some examples, codes 32 may be printed or otherwise embodied on an article different from the article of PPE 30 and attached to PPE 30 using fasteners, such as screws, nails, glue, Velcro, and so forth. While PPE 30 is described as including codes 32, in some examples, articles of equipment 23 may also include codes 32 that include authentication data corresponding to data stored by a distributed ledger.

Each of environments 8 may include one or more physiological sensors 22A-22N (“physiological sensors 22”) configured to detect one or more physiological characteristics of one or more workers 10. Examples of physiological sensors 22 include a heart rate sensor (e.g., configured to detect a pulse or determine a heart rate of worker 10A), breathing sensor (e.g., configured to detect a breathing rate), temperature sensor (e.g., configured to detect a temperature of worker 10A), sweat sensor (e.g., configured to detect how much worker 10A is sweating), among other examples. Physiological sensors 22 generate and output physiological data indicative of the one or more physiological characteristics detected from the one or more workers 10.

In some examples, one or more articles of PPE 30 may include one or more physiological sensors 22. In the example illustrated in FIG. 1, PPE 30A-30N each include one or more physiological sensors 22. For example, PPE 30A may include a smart watch worn by worker 10A, which may include physiological sensor 22A. In some examples, one or more of physiological sensors 22 may be separate from any articles of personal protective equipment. For example, as illustrated in FIG. 1, physiological sensor 22B includes a remote sensor (e.g., an infrared camera that monitors the body temp of one or more workers 10) physically distinct from workers 10 and articles of PPE 30.

Each of environments 8 may include one or more articles of equipment 23A-23N (“equipment 23”). Examples of equipment 23 include vehicles (e.g., trucks, forklifts, cars, etc.), HVAC equipment (e.g., boilers, furnaces, fans, vents, etc.), construction equipment (e.g., drills, saws, etc.), lighting, batteries, or any other piece of equipment. Each article of equipment 23 may be configured to output data indicative of the respective article of equipment 23, such as usage data (e.g., on, off, settings, temperature, run time, etc.). In some examples, equipment 23 includes codes 32 that include authentication data corresponding to data stored by a distributed ledger.

In general, each of environments 8 include computing facilities (e.g., a local area network) by which PPE 30 and/or equipment 23 are able to communicate with PPEMS 6. For example, environments 8 may be configured with wireless technology, such as 602.11 wireless networks, 602.15 ZigBee networks, and the like. In the example of FIG. 1, environment 8B includes a local network 7 that provides a packet-based transport medium for communicating with PPEMS 6 via network 4. In addition, environment 8B includes a plurality of wireless access points 19A, 19B that may be geographically distributed throughout the environment to provide support for wireless communications throughout the work environment.

PPE 30 and/or equipment 23 may be configured to communicate data, such as sensed motions, events and conditions, via wireless communications, such as via 602.11 WiFi protocols, Bluetooth protocol or the like. PPE 30 may, for example, communicate directly with a wireless access point 19. As another example, each worker 10 may be equipped with a respective one of wearable communication hubs 14A-14N that enable and facilitate communication between PPEMS 6 and PPE 30, equipment 23, or both. For example, PPE 30 for the respective worker 10 may communicate with a respective communication hub 14 via Bluetooth or other short-range protocol, and the communication hubs may communicate with PPEMs 6 via wireless communications processed by wireless access points 19. Although shown as wearable devices, hubs 14 may be implemented as stand-alone devices deployed within environment 8B. In some examples, hubs 14 may be articles of PPE.

In general, each of hubs 14 operates as a wireless device for PPE 30 relaying communications to and from the PPE 30 and may be capable of buffering usage data in case communication is lost with PPEMS 6. Moreover, each of hubs 14 is programmable via PPEMS 6 so that local alert rules may be installed and executed without requiring a connection to the cloud. As such, each of hubs 14 provides a relay of streams of usage data from PPE 30 within the respective environment, and provides a local computing environment for localized alerting based on streams of events in the event communication with PPEMS 6 is lost.

As shown in the example of FIG. 1, an environment, such as environment 8B, may also include one or more wireless-enabled beacons, such as beacons 17A-17C, that provide accurate location data within the work environment. For example, beacons 17A-17C may be GPS-enabled such that a controller within the respective beacon may be able to precisely determine the position of the respective beacon. Based on wireless communications with one or more of beacons 17, a given article of PPE 30, or communication hub 14 worn by a worker 10 is configured to determine the location of the worker within work environment 8B. In this way, event or usage data reported to PPEMS 6 may be stamped with positional data to aid analysis, reporting and analytics performed by the PPEMS.

In addition, an environment, such as environment 8B, may also include one or more wireless-enabled sensing stations, such as sensing stations 21A, 21B. Each sensing station 21 includes one or more sensors and a controller configured to output data indicative of sensed environmental conditions. Moreover, sensing stations 21 may be positioned within respective geographic regions of environment 8B or otherwise interact with beacons 17 to determine respective positions and include such positional data when reporting environmental data to PPEMS 6. As such, PPEMS 6 may be configured to correlate the sensed environmental conditions with the particular regions and, therefore, may utilize the captured environmental data when processing event data (also referred to as “usage data”) received from PPE 30 and/or equipment 23. For example, PPEMS 6 may utilize the environmental data to aid generating alerts or other instructions for PPE 30 and for performing predictive analytics, such as determining any correlations between certain environmental conditions (e.g., heat, humidity, visibility) with abnormal worker behavior or increased safety events. As such, PPEMS 6 may utilize current environmental conditions to aid prediction and avoidance of imminent safety events. Example environmental conditions that may be sensed by sensing devices 21 include but are not limited to temperature, humidity, presence of gas, pressure, visibility, wind, precipitation and the like. In general, examples of safety events may include worker activities, conditions or events relating to usage of articles of personal protective equipment (PPE), hazardous environmental conditions, accidental deaths, illness or injury, near-misses, planned or unplanned exposure to hazards, wastage, and costs of accidents. For example, in the context of hearing, vision, or head protection equipment, a safety condition may be such protection equipment being in a standby configuration. In the context of hazardous equipment, a safety condition may be proximity of a worker to the hazardous equipment. Worker illness or injury may include temperature related illness or injury, cardiac related illness or injury, respiratory related illness or injury, or eye or hearing related injury or illness, among other examples. Additional examples of safety events include falls, breathing contaminated air, exposure to radiation or chemicals, etc. Safety events may be categorized in some instance by type, e.g., “worker injuries,” “all safety events,” “exposure,” and so forth.

In example implementations, an environment, such as environment 8B, may also include one or more safety stations 15 distributed throughout the environment to provide viewing stations for accessing PPEMs 6. Safety stations 15 may allow one of workers 10 to check out PPE 30 and/or other safety equipment, verify that safety equipment is appropriate for a particular one of environments 8, and/or exchange data. For example, safety stations 15 may transmit alert rules, software updates, or firmware updates to PPE 30 or other equipment. Safety stations 15 may also receive data cached on PPE 30, hubs 14, and/or other safety equipment. That is, while PPE 30 and/or data hubs 14 may typically transmit usage data to network 4, in some instances, PPE 30, and/or data hubs 14 may not have connectivity to network 4. In such instances, PPE 30, equipment 23, and/or data hubs 14 may store usage data locally and transmit the usage data to safety stations 15 upon being in proximity with safety stations 15. Safety stations 15 may then upload the data from the equipment and connect to network 4.

In addition, each of environments 8 include computing facilities that provide an operating environment for end-user computing devices 16 for interacting with PPEMS 6 via network 4. For example, each of environments 8 typically includes one or more safety managers responsible for overseeing safety compliance within the environment. In general, each user 20 interacts with computing devices 16 to access PPEMS 6. Each of environments 8 may include systems that are described in this disclosure. Similarly, remote users may use computing devices 18 to interact with PPEMS via network 4. For purposes of example, the end-user computing devices 16 may be laptops, desktop computers, mobile devices such as tablets or so-called smart phones and the like.

Users 20, 24 interact with PPEMS 6 to control and actively manage many aspects of safely equipment utilized by workers 10, such as accessing and viewing usage records, analytics and reporting. For example, users 20, 24 may review usage data acquired and stored by PPEMS 6, where the usage data may include data specifying starting and ending times over a time duration (e.g., a day, a week, or the like), data collected during particular events, such as detected falls, sensed data acquired from the user, environment data, and the like. In addition, users 20, 24 may interact with PPEMS 6 to perform asset tracking and to schedule maintenance events for individual pieces of safety equipment, e.g., PPE 30, to ensure compliance with any procedures or regulations. PPEMS 6 may allow users 20, 24 to create and complete digital checklists with respect to the maintenance procedures and to synchronize any results of the procedures from computing devices 16, 18 to PPEMS 6.

Further, as described herein, PPEMS 6 integrates an event processing platform configured to process thousand or even millions of concurrent streams of events from digitally enabled PPE 30, such as SRLs 11 and respirators 13. An underlying analytics engine of PPEMS 6 applies the inbound streams to historical data and models to compute assertions, such as identified safety event signatures which may include anomalies or predicted occurrences of safety events based on conditions or behavior patterns of workers 10. Further, PPEMS 6 provides real-time alerting and reporting to notify workers 10 and/or users 20, 24 of any predicted events, anomalies, trends, and the like.

The analytics engine of PPEMS 6 may, in some examples, process streams of usage data with respect to models to identify relationships or correlations between sensed worker data, environmental conditions, geographic regions and other factors and analyze the impact on safety events. PPEMS 6 may determine, based on the data acquired across populations of workers 10, which particular activities, possibly within certain geographic region, lead to, or are predicted to lead to, unusually high occurrences of safety events.

Moreover, PPEMS 6 generates transactions and updates digital ledger 26 to record detected safety events and trends based on the computed assertions determined from the processing of the streams of event data. In this way, the distributed digital ledger 26 maintained by the managed peer-to-peer network of consensus nodes records safety event information as well as analytical determinations in an immutable, blockchain form.

Further example details of PPEs and worker safety management systems having analytical engines for processing streams of data are described in PCT Patent Application PCT/US2017/039014, filed Jun. 23, 2017, U.S. application Ser. No. 15/190,564, filed Jun. 23, 2016 and U.S. Provisional Application 62/408,634 filed Oct. 14, 2016, the entire content of each of which are hereby expressly incorporated by reference herein.

In this way, PPEMS 6 tightly integrates comprehensive tools for managing personal protection equipment with an underlying analytics engine and communication system to provide data acquisition, monitoring, activity logging, reporting, behavior analytics and alert generation. Moreover, PPEMS 6 provides a communication system for operation and utilization by and between the various elements of system 2. Users 20, 24 may access PPEMS to view results on any analytics performed by PPEMS 6 on data acquired from workers 10. In some examples, PPEMS 6 may present a web-based interface via a web server (e.g., an HTTP server) or client-side applications may be deployed for devices of computing devices 16, 18 used by users 20, 24, such as desktop computers, laptop computers, mobile devices such as smartphones and tablets, or the like.

In some examples, PPEMS 6 may provide a database query engine for directly querying PPEMS 6, which in turn retrieves the information from digital ledger 26, to view acquired safety data, compliance data and any results of the analytic engine, e.g., by the way of dashboards, alert notifications, reports and the like. That is, users 24, 26, or software executing on computing devices 16, 18, may submit queries to PPEMS 6 and receive data corresponding to the queries for presentation in the form of one or more reports or dashboards. Such dashboards may provide various insights regarding system 2, such as baseline (“normal”) operation across worker populations, identifications of any anomalous workers engaging in abnormal activities that may potentially expose the worker to risks, identifications of any geographic regions within environments 2 for which unusually anomalous (e.g., high) safety events have been or are predicted to occur, identifications of any of environments 2 exhibiting anomalous occurrences of safety events relative to other environments, and the like.

As illustrated in detail below, PPEMS 6 may simplify workflows for individuals charged with monitoring and ensure safety compliance for an entity or environment. That is, the techniques of this disclosure may enable active safety management and allow an organization to take preventative or correction actions with respect to certain regions within environments 8, particular articles of PPE or individual workers 10, define and may further allow the entity to implement workflow procedures that are data-driven by an underlying analytical engine.

As one example, the underlying analytical engine of PPEMS 6 may be configured to compute and present customer-defined metrics for worker populations within a given environment 8 or across multiple environments for an organization as a whole. For example, PPEMS 6 may be configured to acquire data and provide aggregated performance metrics and predicted behavior analytics across a worker population (e.g., across workers 10 of either or both of environments 8A, 8B). Furthermore, users 20, 24 may set benchmarks for occurrence of any safety incidences, and PPEMS 6 may track actual performance metrics relative to the benchmarks for individuals or defined worker populations.

As another example, PPEMS 6 may further trigger an alert if certain combinations of conditions are present, e.g., to accelerate examination or service of a safety equipment, such as one of SRLs 11, respirators 13, or the like. In this manner, PPEMS 6 may identify individual pieces of PPE or workers 10 for which the metrics do not meet the benchmarks and prompt the users to intervene and/or perform procedures to improve the metrics relative to the benchmarks, thereby ensuring compliance and actively managing safety for workers 10.

In accordance with techniques of this disclosure, PPEMS 6 is configured to process high-volume streams of PPE data and provides a front-end to allow transaction data to be stored to a secure, immutable distributed ledger maintained by a peer-to-peer network of consensus nodes as one or more blockchains. For example, validation PPEMS 6 may adapt techniques similar to those described in a whitepaper titled “A Next-Generation Smart Contract and Decentralized Application Platform”), available at https://github.com/ethereum/wiki/wiki/White-Paper and incorporated herein by reference in its entirety. Rather than operating as a cryptocurrency, the adaptations described in this disclosure may be used to securely store and authenticate data related to personal protective equipment.

Moreover, PPEMS 6 may perform one or more operations based on data stored in the distributed ledger. In some examples, PPEMS 6 registers one or more article of equipment 23 or PPE 30 with distributed ledger 26. For example, PPEMS 6 may add authentication data (e.g., unique identifier) for an article of PPE 30 to the distributed ledger in blockchain form as managed by the consensus nodes. As another example, PPEMS 6 may associate the authentication data for the article of PPE with a public key corresponding to PPEMS 6. Thus, the transaction data stored to distributed ledger 26 may include the authentication data for a particular article of PPE and a public key corresponding to PPEMS 6. In some examples, the transaction data stored to the distributed ledger includes PPE usage data or sensor data generated by sensing stations 21. For example, PPEMS 6 may store PPE usage data to a distributed ledger, such as a distributed ledger 26 managed by a consensus network. As another example, PPEMS 6 may store at least a portion of the distributed ledger 26. In other words, PPEMS 6 may be a node of the consensus network and may store all or a part of the distributed ledger 26.

Transaction data may include one or more profiles, such as PPE profiles, equipment profiles, environment profiles, hazard profiles, worker profiles, etc. A PPE profile for a respective article of PPE 30 may include PPE usage data for the respective article of PPE 30, such as data generated by one or more sensors of a respective article of PPE 30. PPE profiles may include data indicative of respective articles of PPE 30, such as PPE type, PPE age, or PPE operations. In some examples, PPE profiles include data indicating a respective worker assigned to the PPE 30, data indicating when and/or an amount of time PPE 30 was utilized, whether PPE 30 was utilized correctly, an expected total lifespan of the PPE, an expected remaining lifespan of the PPE, etc. PPE profiles may include data indicating trainings required to operate the PPE, inspection history, or any other data descriptive of PPE or PPE use.

Equipment profiles may include equipment data indicative of one or more articles of equipment. For example, equipment profiles may include equipment data for one or more articles of equipment 23, such as equipment usage data, type of equipment, age of the equipment, indication of remaining lifespan for the equipment (e.g., remaining battery life, filter life, etc.), training required to operate the equipment, among others. Equipment profiles may also include equipment usage data for respective articles of equipment 23, such as when equipment 23 was used, an amount of time equipment 23 was used, and a location where article 23 was used.

In some examples, transaction data stored within the distributed ledger includes environment profiles associated with one or more environments 8. Environment profiles may include environmental data that is generated by one or more sensing stations 21 and is indicative of characteristics of a particular environment 8 (e.g., air temperature, humidity, ambient light, noise levels, radiation, air quality, chemical exposure, visibility, etc.). For example, PPEMS 6 may receive environmental data from one or more sensors of sensing stations 21. In some examples, environmental profiles may include additional environmental data, such as data indicating a location of the environment (e.g., lat/long coordinates, address, city, zipcode, etc.), type of environment (e.g., hospital, factory, warehouse, etc.), size of the environment (e.g., geographical size, number of workers 10, etc.), and the like.

Hazard profiles may include data indicating one or more hazards for a respective environment of environments 8. For example, hazard profiles may include data indicating a type of hazard present within a particular environment 8, a severity of the hazard, quantity of hazards, location of the hazard within the environment, etc.

In some examples, worker profiles may include biographical data (e.g., demographic data, work experience or training data, etc.) for each worker of workers 10. For example, work experience data may include data indicating an amount of time worker 10A has worked for a company, type of worker (e.g., plumber, electrician, surgeon, etc.), or the like. Training data may include data indicating different types of trainings worker 10A has participated in, skills or certifications acquired by worker 10A, or the like. Training data may indicate a past compliance record for a respective worker.

In some examples, PPEMS 6 may store the transaction data (e.g., include PPE profiles, equipment profiles, environment profiles, hazard profiles, and/or worker profiles) to the distributed ledger. For example, PPEMS 6 may store transaction data by sending transaction data to one or more devices on the consensus network such that another device may add transaction data to the distributed ledger, PPEMS 6 may add transaction data to the distributed ledger directly, or a combination therein.

PPEMS 6 performs at least one action based at least in part on the transaction data stored in the blockchain. In some examples, PPEMS 6 may determine whether an article of equipment or article of PPE is authentic based on the transaction data stored within the blockchain. For example, PPEMS 6 may receive data indicative of a particular code 32 corresponding to a particular article of PPE 30. PPEMS 6 may query distributed ledger 26 to determine whether the code 32 has been registered with the blockchain. PPEMS 6 may determine that the article of PPE 30 is authentic in response to determining that the code 32 has been registered with the blockchain. Similarly, PPEMS 6 may determine that the article of PPE 30 is not authentic (e.g., counterfeit) in response to determining that the code has not been registered with the blockchain. PPEMS 6 may output data indicating whether the article of PPE 30 is authentic. For example, PPEMS 6 may output a notification (e.g., to computing devices 16, 18) indicating whether the particular article of PPE 30 is authentic.

In some examples, PPEMS 6 may determine whether a particular article of equipment 23 (e.g., equipment 23A) presents a hazard to a particular worker (e.g., worker 10A) based at least in on transaction data stored within the distributed ledger 26. For example, PPEMS 6 may receive data from PPE 30A indicating a location of PPE 30A and hence a location of worker 10A. Similarly, PPEMS 6 may receive blockchain data 26 indicating a location of the equipment 23A and usage data for equipment 23A indicating the equipment 23A is currently on. In such examples, PPEMS 6 may determine that equipment 23A is in fact operating (e.g., is currently on) because the blockchain data 26 indicates equipment 23 is turned on. In other words, PPEMS 6 may trust that blockchain data 26 is accurate and may use such data in performing actions, even if PPEMS 6 did not receive the data directly from the particular article of equipment 23. In such examples, PPEMS 6 may determine that worker 10A is located proximate to (e.g., within the same environment, the same building, or a threshold distance) equipment 23A. Responsive to determining that worker 10A is located proximate the particular equipment 23, PPEMS 6 may output a notification. For example, PPEMS 6 may output a notification to hub 14A, which may cause hub 14A to generate an alert (e.g., audible, visual, etc.) to worker 10A indicating that worker 10A is proximate to equipment 23A. In some examples, the notification may include data indicating that equipment 23A is dangerous, instructions for the equipment, or an instruction to deactivate (e.g., turn-off) the equipment, among others.

According to aspects of this disclosure, while certain techniques of FIG. 1 are described with respect to PPEMS 6, in other examples, one or more functions may be implemented by hubs 14, PPE 30, or equipment 23. For example, according to aspects of this disclosure, PPEMS 6, hubs 14, PPE 30, and/or equipment 23 may include some or all of the functionality of PPEMS 6. For example, articles of PPE 30, equipment 23, hubs 14, or computing devices 16, 18 may output data to a consensus network. In this way, data generated by PPE 30, equipment 23, hubs 14, or computing devices 16, 18 may be stored within the blockchain managed by the consensus network in approximately real-time, such that the data may be retained in the event of a damage, destruction, theft, or power-loss to PPE 30, equipment 23, hubs 14, or computing devices 16, 18. Further, articles of PPE 30, equipment 23, hubs 14, or computing devices 16, 18 may store at least a portion of a distributed ledger (e.g., blockchain) managed by the consensus network. In other words, articles of PPE 30, equipment 23, hubs 14, or computing devices 16, 18 may be a node of the consensus network. Additionally or alternatively, articles of PPE 30, equipment 23, hubs 14, or computing devices 16, 18 may perform analytics based on the transaction data maintained by the distributed ledger. For example, articles of PPE 30, articles of equipment 23, hubs 14, or computing devices 16, 18 may detect anomalies in the PPE data based at least in part on the transaction data for the distributed ledger and may generate outputs in response to detecting one or more anomalies.

Utilizing a distributed ledger to store data associated with articles of PPE and/or other equipment may provide a more robust, more secure system for storing data about the PPE and other equipment. A computing device verify the authenticity of the PPE and/or PPE usage data based on data stored in a distributed ledger. In this way, the computing device may more accurately indicate whether the PPE and/or PPE usage data is authentic, which may enable workers to verify that the PPE is authentic, which may improve worker safety while utilizing PPE.

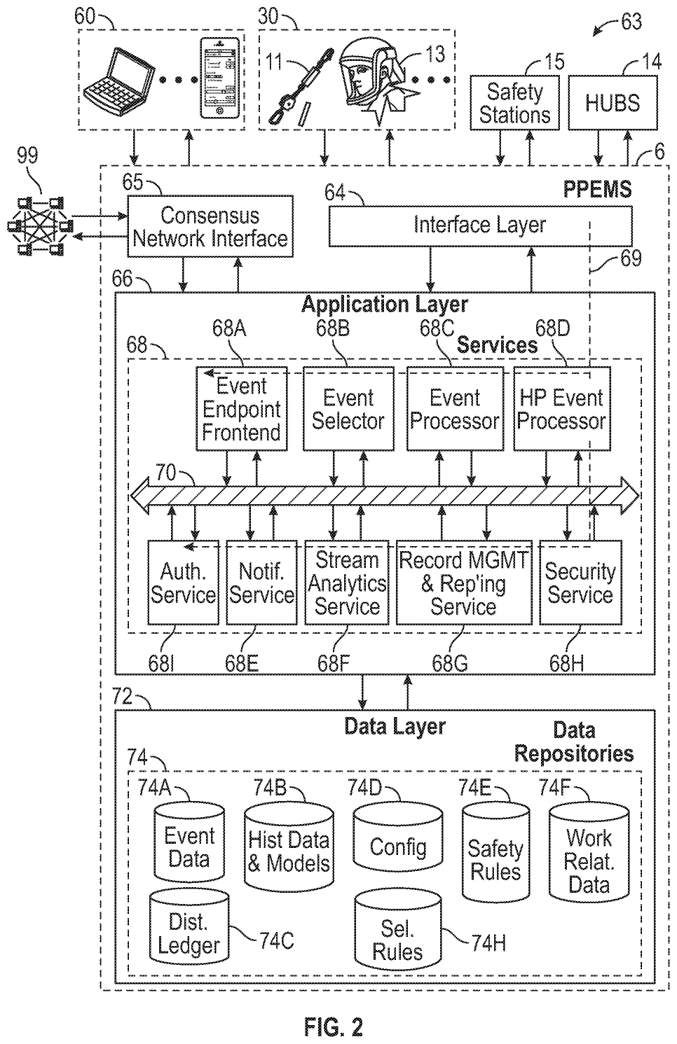

FIG. 2 is a block diagram providing an operating perspective of PPEMS 6 when hosted as cloud-based platform capable of supporting multiple, distinct work environments 8 having an overall population of workers 10 that have a variety of communication enabled PPE 30, such as safety release lines (SRLs) 11, respirators 13, or other safety equipment. In the example of FIG. 2, the components of PPEMS 6 are arranged according to multiple logical layers that implement the techniques of the disclosure. Each layer may be implemented by a one or more modules comprised of hardware, software, or a combination of hardware and software.

In FIG. 2, PPE 30 (e.g., either directly or by way of HUBs 14) as well as computing devices 60, operate as clients 63 that communicate with PPEMS 6 via interface layer 64. Computing devices 60 typically execute client software applications, such as desktop applications, mobile application, and web applications. Computing devices 60 may represent any of computing devices 16, 18 of FIG. 1. Examples of computing devices 60 may include, but are not limited to a portable or mobile computing device (e.g., smartphone, wearable computing device, tablet), laptop computers, desktop computers, smart television platforms, and servers, to name only a few examples.

As further described in this disclosure, PPE 30 communicate with PPEMS 6 (directly or via hubs 14) to provide streams of data acquired from embedded sensors and other monitoring circuitry and receive from PPEMS 6 alerts, configuration and other communications. Client applications executing on computing devices 60 may communicate with PPEMS 6 to send and receive data that is retrieved, stored, generated, and/or otherwise processed by services 68. For instance, the client applications may request and edit safety event data including analytical data stored at and/or managed by PPEMS 6. In some examples, client applications may request and display aggregate safety event data that summarizes or otherwise aggregates numerous individual instances of safety events and corresponding data acquired from PPE 30 and or generated by PPEMS 6. The client applications may interact with PPEMS 6 to query for analytics data about past and predicted safety events, behavior trends of workers 10, to name only a few examples. In some examples, the client applications may output for display data received from PPEMS 6 to visualize such data for users of clients 63. As further illustrated and described in below, PPEMS 6 may provide data to the client applications, which the client applications output for display in user interfaces.

Clients applications executing on computing devices 60 may be implemented for different platforms but include similar or the same functionality. For instance, a client application may be a desktop application compiled to run on a desktop operating system, such as Microsoft Windows, Apple OS X, or Linux, to name only a few examples. As another example, a client application may be a mobile application compiled to run on a mobile operating system, such as Google Android, Apple iOS, Microsoft Windows Mobile, or BlackBerry OS to name only a few examples. As another example, a client application may be a web application such as a web browser that displays web pages received from PPEMS 6. In the example of a web application, PPEMS 6 may receive requests from the web application (e.g., the web browser), process the requests, and send one or more responses back to the web application. In this way, the collection of web pages, the client-side processing web application, and the server-side processing performed by PPEMS 6 collectively provides the functionality to perform techniques of this disclosure. In this way, client applications use various services of PPEMS 6 in accordance with techniques of this disclosure, and the applications may operate within various different computing environment (e.g., embedded circuitry or processor of a PPE, a desktop operating system, mobile operating system, or web browser, to name only a few examples).

As shown in FIG. 2, PPEMS 6 includes an interface layer 64 that represents a set of application programming interfaces (API) or protocol interface presented and supported by PPEMS 6. Interface layer 64 initially receives messages from any of clients 63 for further processing at PPEMS 6. Interface layer 64 may therefore provide one or more interfaces that are available to client applications executing on clients 63. In some examples, the interfaces may be application programming interfaces (APIs) that are accessible over a network. Interface layer 64 may be implemented with one or more web servers. The one or more web servers may receive incoming requests, process and/or forward data from the requests to services 68, and provide one or more responses, based on data received from services 68, to the client application that initially sent the request. In some examples, the one or more web servers that implement interface layer 64 may include a runtime environment to deploy program logic that provides the one or more interfaces. As further described below, each service may provide a group of one or more interfaces that are accessible via interface layer 64.

In some examples, interface layer 64 may provide Representational State Transfer (RESTful) interfaces that use HTTP methods to interact with services and manipulate resources of PPEMS 6. In such examples, services 68 may generate JavaScript Object Notation (JSON) messages that interface layer 64 sends back to the client application 61 that submitted the initial request. In some examples, interface layer 64 provides web services using Simple Object Access Protocol (SOAP) to process requests from client applications. In still other examples, interface layer 64 may use Remote Procedure Calls (RPC) to process requests from clients 63. Upon receiving a request from a client application to use one or more services 68, interface layer 64 sends the data to application layer 66, which includes services 68.

As shown in FIG. 2, PPEMS 6 also includes an application layer 66 that represents a collection of services for implementing much of the underlying operations of PPEMS 6. Application layer 66 receives data included in requests received from client applications and further processes the data according to one or more of services 68 invoked by the requests. Application layer 66 may be implemented as one or more discrete software services executing on one or more application servers, e.g., physical or virtual machines. That is, the application servers provide runtime environments for execution of services 68. In some examples, the functionality interface layer 64 as described above and the functionality of application layer 66 may be implemented at the same server.

Application layer 66 may include one or more separate software services 68, e.g., processes that communicate, e.g., via a logical service bus 70 as one example. Service bus 70 generally represents a logical interconnections or set of interfaces that allows different services to send messages to other services, such as by a publish/subscription communication model. For instance, each of services 68 may subscribe to specific types of messages based on criteria set for the respective service. When a service publishes a message of a particular type on service bus 70, other services that subscribe to messages of that type will receive the message. In this way, each of services 68 may communicate data to one another. As another example, services 68 may communicate in point-to-point fashion using sockets or other communication mechanism. In still other examples, a pipeline system architecture could be used to enforce a workflow and logical processing of data a messages as they are process by the software system services. Before describing the functionality of each of services 68, the layers is briefly described herein.

Data layer 72 of PPEMS 6 represents a data repository that provides persistence for data in PPEMS 6 using one or more data repositories 74. A data repository, generally, may be any data structure or software that stores and/or manages data. Examples of data repositories include but are not limited to relational databases, multi-dimensional databases, maps, and hash tables, to name only a few examples. Data layer 72 may be implemented using Relational Database Management System (RDBMS) software to manage data in data repositories 74. The RDBMS software may manage one or more data repositories 74, which may be accessed using Structured Query Language (SQL). Data in the one or more databases may be stored, retrieved, and modified using the RDBMS software. In some examples, data layer 72 may be implemented using an Object Database Management System (ODBMS), Online Analytical Processing (OLAP) database or other suitable data management system.

As shown in FIG. 2, each of services 68A-68J (“services 68”) is implemented in a modular form within PPEMS 6. Although shown as separate modules for each service, in some examples the functionality of two or more services may be combined into a single module or component. Each of services 68 may be implemented in software, hardware, or a combination of hardware and software. Moreover, services 68 may be implemented as standalone devices, separate virtual machines or containers, processes, threads or software instructions generally for execution on one or more physical processors.

In some examples, one or more of services 68 may each provide one or more interfaces that are exposed through interface layer 64. Accordingly, client applications of computing devices 60 may call one or more interfaces of one or more of services 68 to perform techniques of this disclosure.

Services 68 may include an event processing platform including an event endpoint frontend 68A, event selector 68B, event processor 68C and high priority (HP) event processor 68D. Event endpoint frontend 68A operates as a front end interface for receiving and sending communications to PPE 30 and hubs 14. In other words, event endpoint frontend 68A operates to as a front line interface to safety equipment deployed within environments 8 and utilized by workers 10. In some instances, event endpoint frontend 68A may be implemented as a plurality of tasks or jobs spawned to receive individual inbound communications of event streams 69 from the PPE 30 carrying data sensed and captured by sensors for a worker, PPE, and/or work environment. When receiving event streams 69, for example, event endpoint frontend 68A may spawn tasks to quickly enqueue an inbound communication, referred to as an event, and close the communication session, thereby providing high-speed processing and scalability. Each incoming communication may, for example, carry recently captured data representing sensed conditions, motions, temperatures, actions or other data, generally referred to as events. Communications exchanged between the event endpoint frontend 68A and the PPEs may be real-time or pseudo real-time depending on communication delays and continuity.

Event selector 68B operates on the stream of events 69 received from PPE 30 and/or hubs 14 via frontend 68A and determines, based on rules or classifications, priorities associated with the incoming events. Based on the priorities, event selector 68B enqueues the events for subsequent processing by event processor 68C or high priority (HP) event processor 68D. Additional computational resources and objects may be dedicated to HP event processor 68D so as to ensure responsiveness to critical events, such as incorrect usage of PPEs, use of incorrect filters and/or respirators based on geographic locations and conditions, failure to properly secure SRLs 11 and the like. Responsive to processing high priority events, HP event processor 68D may immediately invoke notification service 68E to generate alerts, instructions, warnings or other similar messages to be output to SRLs 11, hubs 14 and/or remote users 20, 24. Events not classified as high priority are consumed and processed by event processor 68C.

In general, event processor 68C or high priority (HP) event processor 68D operate on the incoming streams of events to update event data 74A within data repositories 74. In general, event data 74A may include all or a subset of usage data obtained from PPE 30. For example, in some instances, event data 74A may include entire streams of samples of data obtained from electronic sensors of PPE 30. In other instances, event data 74A may include a subset of such data, e.g., associated with a particular time period or activity of PPE 30. Event processors 68C, 68D may create, read, update, and delete event data stored in event data 74A. Event data for may be stored in a respective database record as a structure that includes name/value pairs of data, such as data tables specified in row/column format. For instance, a name (e.g., column) may be “worker ID” and a value may be an employee identification number. An event record may include data such as, but not limited to: worker identification, PPE identification, acquisition timestamp(s) and data indicative of one or more sensed parameters.

In addition, event selector 68B directs the incoming stream of events (e.g., usage data or event data) to stream analytics service 68F, which represents an example of an analytics engine configured to perform in depth processing of the incoming stream of events to perform real-time analytics. Stream analytics service 68F may, for example, be configured to process and compare multiple streams of event data 74A with historical data and models 74B in real-time as event data 74A is received. In this way, stream analytic service 68D may be configured to detect safety event signatures (e.g., anomalies, patterns, and the like), transform incoming event data values, trigger alerts upon detecting safety concerns based on conditions or worker behaviors. Historical data and models 74B may include, for example, specified safety rules, business rules and the like. In this way, historical data and models 74B may characterize activity of a user of SRL 11, e.g., as conforming to the safety rules, business rules, and the like. In addition, stream analytic service 68D may generate output for communicating to PPPE 30 by notification service 68F or computing devices 60 by way of record management and reporting service 68D.

Analytics service 68F may process inbound streams of events, potentially hundreds or thousands of streams of events, from enabled safety PPE 30 utilized by workers 10 within environments 8 to apply historical data and models 74B to compute assertions, such as identified safety event signatures, anomalies or predicted occurrences of imminent safety events based on conditions or behavior patterns of the workers. Analytics service 68D may publish the assertions to notification service 68F and/or record management by service bus 70 for output to any of clients 63. In some examples, at least one sensor that generates usage data that characterizes at least a worker associated with the article of PPE or a work environment; and to detect the safety event signature in the stream of usage, analytics service 68F processes the usage data that characterizes the worker associated with the article of PPE or the work environment.

In this way, analytics service 68F may be configured as an active safety management system that predicts imminent safety concerns and provides real-time alerting and reporting. In addition, analytics service 68F may be a decision support system that provides techniques for processing inbound streams of event data to generate assertions in the form of statistics, conclusions, and/or recommendations on an aggregate or individualized worker and/or PPE basis for enterprises, safety officers and other remote users. For instance, analytics service 68F may apply historical data and models 74B to determine, for a particular worker, the likelihood that a safety event is imminent for the worker based on detected behavior or activity patterns, environmental conditions and geographic locations. In some examples, analytics service 68F may determine whether a worker is currently impaired, e.g., due to exhaustion, sickness or alcohol/drug use, and may require intervention to prevent safety events. As yet another example, analytics service 68F may provide comparative ratings of workers or type of safety equipment in a particular environment 8.

Hence, analytics service 68F may maintain or otherwise use one or more models that provide risk metrics to predict safety events. Analytics service 68F may also generate order sets, recommendations, and quality measures. In some examples, analytics service 68F may generate user interfaces based on processing data stored by PPEMS 6 to provide actionable data to any of clients 63. For example, analytics service 68F may generate dashboards, alert notifications, reports and the like for output at any of clients 63. Such data may provide various insights regarding baseline (“normal”) operation across worker populations, identifications of any anomalous workers engaging in abnormal activities that may potentially expose the worker to risks, identifications of any geographic regions within environments for which unusually anomalous (e.g., high) safety events have been or are predicted to occur, identifications of any of environments exhibiting anomalous occurrences of safety events relative to other environments, and the like.

Although other technologies can be used, in one example implementation, analytics service 68F utilizes machine learning when operating on streams of safety events so as to perform real-time analytics. That is, analytics service 68F includes executable code generated by application of machine learning to training data of event streams and known safety events to detect patterns. The executable code may take the form of software instructions or rule sets and is generally referred to as a model to which event streams 69 can be applied for detecting similar patterns and predicting upcoming events.

Analytics service 68F may, in some example, generate separate models for a particular worker, a particular population of workers, one or more articles of PPE or types of PPE, a particular environment, or combinations thereof. Analytics service 68F may update the models based on usage data received from PPE 30. For example, analytics service 68F may update the models for a particular worker, a particular population of workers, one or more articles of PPE or types of PPE a particular environment, or combinations thereof based on data received from PPE 30.

In some examples, analytics service 68F store at least a portion of a stream of usage data and at least one model for detecting a safety event signature. In some examples, the stream of usage data comprises metrics for a plurality of articles of PPE, workers, and/or work environments. As described in this disclosure, at least one model is trained based as least in part on a set of usage data generated, prior to receiving the stream of usage data, by one or more other articles of PPE of a same type as the article of PPE.

In some examples the “same type” may refer to identical but separate instances of PPE. In other examples the “same type” may not refer to identical instances of PPE. For instance, although not identical, a same type may refer to PPE in a same class or category of PPE, same model of PPE, or same set of one or more shared functional or physical characteristics, to name only a few examples. Similarly, a same type of work environment or worker may refer to identical but separate instances of work environment types or worker types. In other examples, although not identical, a same type may refer to a worker or work environment in a same class or category of worker or work environment or same set of one or more shared behavioral, physiological, environmental characteristics, to name only a few examples.

In some examples, safety event signature comprises at least one of an anomaly in a set of usage data, a pattern in a set of usage data, a particular set of occurrences of particular events over a defined period of time, a particular set of types of particular events over a defined period of time, a particular set of magnitudes of particular events over a defined period of time, or a value that satisfies a threshold (e.g., greater than, equal to, or less than). In some examples, the threshold is hard-coded, machine generated, and/or user-configurable. In some examples, a safety event signature may be a unique or a particularly defined profile of a set of events. In some examples, each respective event is generated at a same defined interval, wherein each respective event includes a respective set of values that correspond to a same set of defined metrics, and/or wherein respective sets of values in different respective events are different. Examples of a defined interval (which may be hard-coded, user-configurable, and/or machine-generated) include: 500 milliseconds, 1 minute, 5 minutes, 10 minutes, an interval in a range between 0-30 seconds, an interval in a range between 0-5 minutes, an interval in a range between 0-10 minutes, an interval in a range between 0-30 minutes, an interval in a range between 0-60 minutes, an interval in a range between 0-12 hours. In some examples, the set of defined metrics comprises one or more of a timestamp, characteristics of the article of PPE, characteristics of a worker associated with the article of PPE, or characteristics a work environment.

In some examples, analytics service 68F detects a safety event signature in a stream of usage data based on processing the stream of usage data with the model. To process the stream of usage data with the model, analytics service 68F may apply the usage data to the model. To apply the usage data to the model, analytics service 68F may generate a structure, such as a feature vector, in which the usage data is stored. The feature vector may include a set of values that correspond to metrics (e.g., characterizing PPE, worker, work environment, to name a few examples), where the set of values are included in the usage data. The model may receive the feature vector as input, and based on one or more relations defined by the model (e.g., probabilistic, deterministic or other functions within the knowledge of one of ordinary skill in the art) that has been trained, the model may output one or more probabilities or scores that indicate likelihoods of safety events based on the feature vector. Based on the safety event signature, analytics service 68F may generate an output in response. In some examples, at least one safety rule is mapped to at least one safety event, the at least one safety event is mapped to the safety event signature, and/or the safety event signature corresponds to at least the portion of a stream of usage data. As such, if at least a portion of a stream of usage data corresponds to a safety event signature, analytics service 68F may test and/or execute one or more safety rules that correspond to the safety event mapped to the safety event signature. In some examples, at least the portion of the stream of usage data is deleted after the one or more computer processors detect the safety event signature. For instance, the portion of the stream of usage data may be deleted after a threshold amount of time, or after being processed to detect the safety event signature.

In some examples, to generate output in response to detecting a safety event signature, analytics service 68F may cause one or more components of PPEMS 6 to send a notification to at least one of the article of PPE, a hub associated with a user and configured to communicate with the article of PPE and at least one remote computing device, or a computing device associated with person who is not the user. In some examples, to generate the output in response to detecting the safety event signature, analytics service 68F may cause one or more components of PPEMS 6 to send a notification that alters an operation of the article of PPE. In some examples, to generate output in response to detecting the safety event signature, analytics service 68F may cause one or more components of PPEMS 6 to output for display a user interface that indicates the safety event in association with at least one of a user, work environment, or the article of PPE. In some examples, to generate an output in response to detecting the safety event signature, the one or more processors may generate a user interface that is based at least in part on a safety event that corresponds to the safety event signature. In some examples, the user interface includes at least one input control that requires a responsive user input within a threshold time period, and in response to the threshold time period expiring without the responsive user input, PPEMS 6 may perform at least one operation based at least in part on the threshold time period expiring without the responsive user input. In some examples, an article of PPE comprises at least one of an air respirator system, a fall protection device, a hearing protector, a head protector, a garment, a face protector, an eye protector, a welding mask, or an exosuit.

In some examples, prior to detection of a safety event signature, analytics service 68F may determine, based at least in part on a data stream of usage data, that an article of PPE is operating in a normal state. A normal state may be a predefined state based on user input and/or machine-generated based on determined steady-state or acceptable conditions or use. In response to detection of a detection of the safety event signature, analytics service 68F may determine that the article of PPE is not operating in the normal state. For instance, prior to detecting the safety event signature, the article of PPE (or worker and/or worker environment) may have been operating in a steady-state or acceptable condition, which was subsequently followed by a safety event signature indicating an abnormal state or state other than the normal state. In some examples, a portion of a stream of usage data is a first portion of the stream of usage data, a safety event signature is a first safety event signature, a normal state corresponds to a second safety event signature, the first portion of the data stream corresponds to the first safety event signature, and a second portion of the data stream corresponds to the second safety event signature.

In some examples, a set of articles of PPE are associated with a user. Each article of PPE in the set of articles of PPE includes a motion sensor, such as an accelerometer, gyroscope or other device that can detect motion. Analytics service 68F may receive a respective stream of usage data from each respective motion sensor of each respective article of PPE of the set of articles of PPE. To detect a safety event signature, analytics service 68F may detect a safety event signature corresponding to a relative motion that is based at least in part on the respective stream of usage data from each respective motion sensor. That is, based on multiple different streams of usage data from different motion sensors positioned at different locations on the same user, analytics service 68F may determine a relative motion of the worker. In some examples, the safety event signature corresponds to a safety event that indicates ergonomic stress, and in some examples, analytics service 68F may determine that the ergonomic stress satisfies a threshold (e.g., greater than or equal to the threshold).

Alternatively, or in addition, analytics service 68F may communicate all or portions of the generated code and/or the machine learning models to hubs 14, PPE 30, or equipment 23 for execution thereon so as to provide local alerting in near-real time to PPEs. Example machine learning techniques that may be employed to generate models 74B can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbour (kNN), Learning Vector Quantization (LUQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

Record management and reporting service 68G processes and responds to messages and queries received from computing devices 60 via interface layer 64. For example, record management and reporting service 68G may receive requests from client computing devices for event data related to individual workers, populations or sample sets of workers, geographic regions of environments 8 or environments 8 as a whole, individual or groups/types of PPE 30. In response, record management and reporting service 68G accesses event data based on the request. Upon retrieving the event data, record management and reporting service 68G constructs an output response to the client application that initially requested the data. In some examples, the data may be included in a document, such as an HTML document, or the data may be encoded in a JSON format or presented by a dashboard application executing on the requesting client computing device. For instance, as further described in this disclosure, example user interfaces that include the event data are depicted in the figures.

As additional examples, record management and reporting service 68G may receive requests to find, analyze, and correlate PPE event data. For instance, record management and reporting service 68G may receive a query request from a client application for event data 74A over a historical time frame, such as a user can view PPE event data over a period of time and/or a computing device can analyze the PPE event data over the period of time.

In example implementations, services 68 may also include security service 68H that authenticate and authorize users and requests with PPEMS 6. Specifically, security service 68H may receive authentication requests from client applications and/or other services 68 to access data in data layer 72 and/or perform processing in application layer 66. An authentication request may include credentials, such as a username and password. Security service 68H may query security data in data layer 72 to determine whether the username and password combination is valid. Configuration data 74D may include security data in the form of authorization credentials, policies, and any other data for controlling access to PPEMS 6. As described above, security data in data layer 72 may include authorization credentials, such as combinations of valid usernames and passwords for authorized users of PPEMS 6. Other credentials may include device identifiers or device profiles that are allowed to access PPEMS 6.

In the example of FIG. 2, a safety manager may initially configure one or more safety rules. As such, remote user 24 may provide one or more user inputs at computing device 18 that configure a set of safety rules for work environment 8A and 8B. For instance, a computing device 60 of the safety manager may send a message that defines or specifies the safety rules. Such message may include data to select or create conditions and actions of the safety rules. PPEMS 6 may receive the message at interface layer 64 which forwards the message to rule configuration component 68I. Rule configuration component 68I may be combination of hardware and/or software that provides for rule configuration including, but not limited to: providing a user interface to specify conditions and actions of rules, receive, organize, store, and update rules included in safety rules data store 74E.

Safety rules data store 74E may be a data store that includes data representing one or more safety rules. Safety rules data store 74E may be any suitable data store such as a relational database system, online analytical processing database, object-oriented database, or any other type of data store. When rule configuration component 68I receives data defining safety rules from computing device 60 of the safety manager, rule configuration component 68I may store the safety rules in safety rules data store 74E.

In the example of FIG. 2, PPEMS 6 also includes work relation data 74F. Work relation data 74F may include mappings between data that corresponds to PPE, workers, and work environments. Work relation data 74F may be any suitable datastore for storing, retrieving, updating and deleting data. RMRS 68G may store a mapping between the unique identifier of worker 10A and a unique device identifier of data hub 14A. Work relation data store 74F may also map a worker to an environment.

According to aspects of this disclosure, authentication service 68I may receive event streams 69 and may store transaction data to a distributed ledger (e.g., blockchain) managed by a consensus network. In some examples, authentication service 68I may output the data received from hubs 14, PPE 30, equipment 23, or sensing stations 21 to the consensus network for storing to a distributed ledger. For example, authentication service 68I may broadcast or output data received from hubs 14, PPE 30, equipment 23, or sensing stations 21 via consensus network interface 65 to store transactions of data to the distributed ledger maintained by consensus network 99. Each of the computing devices of consensus network 99 may receive the data via consensus network interface 65 and may store a copy of the data from PPEMS 6 in a local data store on the respective nodes of consensus network 99. In this way, consensus network 99 may maintain an immutable, secure ledger of data received from PPEMS 6.

In some examples, distributed ledger data store 74C stores all or part of the ledger managed by the consensus network. For example, PPEMS 6 may be a node of the consensus network 99 such that distributed ledger 74C may include a local copy of all or part of a ledger (e.g., blockchain) while other nodes of the consensus network 99 also store copies of the ledger. In some examples, rather than storing a local copy of the ledger, distributed ledger data store 74C includes data indicative of the distributed ledger, such as data representative of the state of the distributed ledger at a particular point in time.

Authentication service 68I performs one or more actions based on the transaction data stored within the distributed ledger managed by the consensus network. In some examples, the one or more actions includes determining whether an article of equipment 23 or article of PPE 30 is authentic based on the transaction data stored within the distributed ledger. In some examples, the manufacturer of the article of PPE may register a code 32 corresponding to an article of PPE with the distributed ledger. For example, the manufacturer may have rights or a public/private key to register such codes while other users of the distributed ledger may not have such rights/keys, which may prevent unauthorized users from registering counterfeit articles or counterfeit codes with the distributed ledger. The manufacturer may send transaction data including authentication data for one or more articles of PPE to consensus network 99, such that each node of consensus network 99 may include data indicative of a code 32 for the articles of PPE within the respective local copies of the distributed ledger.

Authentication service 68I may confirm ownership of a particular item of PPE and/or origin of manufacture based on the transaction data within the ledger. As one example, a user of PPEMS 6 may scan a code 32 corresponding to a particular article of PPE 30 and authentication service 68I may receive data indicative of the particular code 32 corresponding to a particular article of PPE 30. Authentication service 68I may query a local copy of the distributed ledger by querying distributed ledger data store 74C to determine whether the code 32 has been registered with the distributed ledger. As another example, authentication service 68I may query the nodes of consensus network 99 by sending, via consensus network interface 65, a request to authenticate the code 32 to each of the nodes of consensus network 99. Authentication service 68I may determine that the article of PPE 30 is authentic in response to determining that the code 32 has been registered with the distributed ledger data store 74C, or in response to receiving data from consensus network 99 that the code 32 is registered with a majority of the nodes of consensus network 99. Similarly, authentication service 68I may determine that the article of PPE 30 is not authentic (e.g., counterfeit) in response to determining that the code 32 has not been registered with the distributed ledger data store 74C, or in response to receiving data from consensus network 99 that code 32 is not registered with a majority of the nodes of consensus network 99. PPEMS 6 may output data indicating whether the article of equipment 23 or PPE 30 is authentic. For example, notification service 68E may output a notification (e.g., to computing devices 60) indicating whether or not the particular article of equipment or PPE is authentic.

Authentication service 68I may determine whether the constituent components of a particular article of equipment 23 or PPE 30 are authentic. For example, manufacturers of constituent components may register a code associated with each respective component to the distributed ledger. Upon receiving the constituent components, a user (e.g., worker or assembler of constituent components) may scan a code 32 corresponding to the constituent components and may send an indication of the code 32 to PPEMS 6. PPEMS 6 may determine whether the codes 32, and hence articles of equipment or PPE 30, are authentic based on data stored within distributed ledger data store 74C or by querying the nodes of consensus network 99.

In some examples, the actions performed by authentication service 68I includes tracking the PPE history and validating the PPE history. For example, an article of PPE 30 or equipment 23 may send usage data to the consensus network directly (e.g., articles of PPE 30 or equipment 23 may interface with consensus network 99 without sending data to PPEMS 6) or indirectly (e.g., via PPEMS 6). In some examples, PPE 30 sends usage data to the consensus network automatically, such that the PPE 30 sends usage data to the consensus network as the article of PPE 30 or equipment 30 is moved between work environments 8 or is utilized by different users (e.g., different workers 10 of the same company, or different users when the article of PPE 30 or equipment 23 is rented or sold). For example, PPE 30 may send PPE generated usage data to be stored by a distributed ledger managed by a manufacturer of the PPE 30. Similarly, a user of PPE 30 may store additional usage data to the same distributed ledger or a different distributed ledger. Authentication service 68I may verify the authenticity of the additional PPE usage data based on the PPE generated usage data that is stored within a manufacturer distributed ledger maintained by manufacturers of PPE (e.g., the nodes of consensus network 99 correspond to various PPE manufacturers). For example, distributed ledger data store 74C may include a local copy of the manufacturer ledger and authentication service 68I may query the local copy of the manufacturer ledger. As another example, authentication service 68I may query consensus network 99 via consensus network interface 65. For example, authentication service 68I may determine that the additional PPE usage data is authentic in response to determining that the additional PPE data corresponds to (e.g., matches) the PPE generated usage data or receiving data from a majority of consensus network 99 indicating that the additional PPE data corresponds to the PPE generated usage data. Similarly, authentication service 68I may determine that the additional PPE usage data is not authentic in response to determining that the additional PPE data does not correspond to (e.g., match) the PPE generated usage data or receiving data from a majority of consensus network 99 indicating that the additional PPE data does not correspond to the PPE generated usage data. Similarly, authentication service 68I.

Authentication service 68I may determine an amount of usage of an article of PPE 30 and a difference in condition of the article of PPE 30 (e.g., which may indicate PPE 30 been damaged) based on the transaction data. For example, usage data associated with the article of PPE 30 that is stored within the distributed ledger (e.g., distributed ledger data store 74C) may indicate a current user (e.g., worker, person renting the PPE, etc.) of a particular article of PPE 30. As another example, the usage data may indicate a condition of the PPE at a first time, such as whether the PPE 30 has any damage (e.g., and if so, characterizing the damage), or an amount of usable life of the PPE or a component of the PPE (e.g., filter or battery life). In some examples, the usage data may indicate the condition of the PPE at a first time (e.g., when the PPE is received by a first user, such as a renter) and the condition of the PPE at a second time (e.g., when the PPE is delivered to another user, such as being returned to the owner). Thus, authentication service 68I may receive an indication of the current condition of the article of PPE 30, determine the condition of the article of PPE at an earlier time based on a local copy of the distributed ledger or querying consensus network 99, determine a difference in the condition of the article of PPE 30, and output a notification in response to determining that the condition has changed. In some examples, the notification may include an indication of the difference in condition.

Authentication service 68I may determine whether PPE usage data is authentic based at least in part transaction data stored within the distributed ledger (e.g., a local copy stored within data store 74C or by querying consensus network 99). In some examples, distributed ledger data store 74C may include distributed ledger data for a plurality of distributed ledgers managed by different types of entities (e.g., different consensus networks 99). For example, distributed ledger data store 74C may include distributed ledger data for a first distributed ledger managed by a consensus network of PPE manufactures, and distributed ledger data for a second distributed ledger managed by a consensus network of employers of workers 10. PPE 30A may send the PPE data to a manufacturer of PPE 30A and an employer of worker 10A, where the manufacturer and employer may each publish the PPE data to respective distributed ledgers. Authentication service 68I may receive data associated with the manufacturer and employer distributed ledgers 26 determine whether the PPE data is authentic based on querying distributed ledger data store 74C or consensus network 99. In some examples, authentication service 68I may determine that the PPE data for PPE 30A is not authentic (e.g., is counterfeit) in response to determining the PPE data in the employer distributed ledger does not correspond to or is inconsistent with the PPE data in the manufacturer distributed ledger. In such examples, authentication service 68I may output a notification indicating the PPE data for PPE 30A is not authentic. For example, authentication service 68I may output a notification (e.g., text message, email, etc.) to one or more of computing devices (e.g., computing devices 16, 18) indicating the PPE data is not authentic or is inconsistent.

Authentication service 68I may validate inspection data for PPE 30, equipment 23, or environment 8 based on the transaction data in the distributed ledger. In some examples, authentication service 68I may determine whether inspections of PPE 30 are being performed properly. For example, authentication service 68I may determine inspections are performed properly when the inspections are performed according to a pre-defined schedule, by an authorized service provider, or in a pre-defined order. For example, users of computing devices 16, 18 may include authorized service providers who may have rights to register inspection data or maintenance data to the consensus network. In some examples, authentication service 68I may query the distributed ledger data store 74C or consensus network 99 to identify a type of inspection performed, who performed the inspection, what was inspected, inspection results, etc. Authentication service may query the distributed ledger data store 74C or consensus network 99 to determine whether an inspection was performed at appropriate times (e.g., according to a predefined schedule) or are performed by authorized entities. For example, authentication service 68I may determine whether the inspection data was performed by an authorized entity based on determining whether the data was digitally signed with a public key for the authorized entity.

Authentication service 68I may validate maintenance data for PPE 30 or equipment 23 based on transaction data within the distributed ledger managed by the consensus network. In some examples, authentication service 68I may determine whether maintenance of PPE 30 is being performed properly. For example, authentication service 68I may determine maintenance is performed properly when the maintenance is performed according to a pre-defined schedule, or by an authorized service provider. In some examples, authentication service 68I may query the distributed ledger data store 74C or consensus network 99 to retrieve maintenance data, such as when maintenance was performed, a type of maintenance performed (e.g., cleaning, or new parts such as new filters, batteries, etc.), a maintenance schedule (e.g., when to clean, change filters, take the equipment/PPE out of service, etc.), cost of parts or work performed, etc. Authentication service 68I may determine that the maintenance is performed properly in response to determining the maintenance was performed according to a maintenance schedule or by an authorized entities.

As another example, authentication service 68I may determine whether maintenance or inspection data stored to the distributed ledger by a user (e.g., worker or authorized service provider) corresponds to maintenance or inspection data generated by PPE 30 or equipment 23. For example, PPE 30 may generate maintenance data in response to detecting PPE 30 has been cleaned, reset, or received a new component (e.g., a new filter, battery, etc.) and may broadcast the maintenance data to consensus network 99 (e.g., and store the maintenance data within the local distributed ledger data store 74C). Similarly, a worker 10A may store, to consensus network 99, maintenance data to indicating he/she performed maintenance on PPE 30 and a type of maintenance performed. Authentication service 68I may determine whether the maintenance data provided by the worker corresponds to (e.g., matches) maintenance data generated by PPE 30 by querying consensus network 99 or a local copy of the distributed ledger maintained by consensus network 99. In response to determining that the maintenance data provided by the worker does not correspond to the maintenance data generated by PPE 30, notification service 68E may output a notification indicating at least some of the maintenance data may be invalid. Authentication service 68I may determine whether a user of PPE 30 (e.g., worker 10A or an authorized service provider) is authorized to perform the maintenance by determining whether the user has rights to register inspection data to the consensus network. PPEMS 6 may determine whether the user is authorized to perform the maintenance based on determining whether the maintenance data is digitally signed with a public key for the authorized maintenance entity. In some examples, PPEMS 6 may determine a cost of the maintenance based on the maintenance data within the distributed ledger and may output a notification (e.g., to a user of computing devices 16, 18) indicating a worker 10A who performed the maintenance and an amount to reimburse worker 10A.

In some examples, authentication service 68I may aggregate usage data for different groups of PPE 30 or equipment 23 and may store usage data for the different groups of PPE 30 or equipment 23 to separate distributed ledgers (e.g., maintained by different consensus networks 99). For example, authentication service 68I may determine a type of PPE 30 or equipment 23 and may store usage data to a distributed ledger associated with that type of PPE 30 or equipment 23 (e.g. by sending the data to a consensus network 99 associated with that type of PPE 30 or equipment 23). In some examples, analytics service 68I may determine a general type for each PPE 30 or equipment 23, such as breathing devices, fall protection devices, apparel, etc., or a more specific type such as respirator, self-retracting lifeline, eyewear, etc. As another example, authentication service 68I may determine a type of PPE 30 or equipment 23 based on manufacturer, model number, etc. Responsive to determining a type of PPE 30 or equipment 23, authentication service 68I may store the usage data to a distributed ledger corresponding to the type of the respective PPE 30 or equipment 23.

In some examples, stream analytic service 68I validates data stored within the distributed ledger managed by the consensus network based on other data that is also stored within the distributed ledger managed by the consensus network. For example, PPEMS 6 may write workflows or rules to the distributed ledger maintained by consensus network 99 (e.g., and may store a local copy of the distributed ledger within data store 74C) and may determine whether the workflows and/or rules are satisfied based on data written to the distributed ledger maintained by consensus network 99. For example, authentication service 68I may determine whether first data written to the distributed ledger is consistent with second data written to the distributed ledger (e.g., by querying consensus network 99 or a local copy of the distributed ledger stored within data store 74C). According to one scenario, the distributed ledger may include worker data (e.g., worker training data uploaded by one of workers 10 via one of computing devices 16, 18) in a distributed ledger and worker training data (e.g., uploaded by a user performing the trainings) in a (e.g., different) distributed ledger. Authentication service 68I may determine whether the worker data is consistent with the worker training data based on transaction data within distributed ledger. For example, authentication service may compare a worker training history provided by the worker with the worker training data uploaded by the trainer. Authentication service 68I may determine whether the training data provided by worker 10A (e.g., indicating whether worker 10A completed a training or performed to a particular level of proficiency) corresponds to training data provided by the trainer. Responsive to determining that the training data provided by worker 10A does not correspond to the training data provided by the trainer, authentication service 68I may output a notification indicating a difference in the training data. In some examples, the notification may indicate a restriction on PPE 30 or equipment 23 that worker 10A is authorized to use or other restrictions upon worker 10A, which may be put into place until a cause of the difference is determined or the difference is rectified.

Authentication service 68I may determine whether worker 10A has participated in one or more trainings based on worker data stored within the distributed ledger. For example, worker 10A may transition from working for a first employer to working for a second, different employer. The second employer may require the worker to receive a training that worker 10A has already received at the first employer. According to techniques of this disclosure, each employer may store worker data, such as a worker profile, to the distributed ledger. In some examples, each employer may store certain worker data (e.g., all or a portion of a worker profile) in a first, private ledger and other worker data (e.g., a subset of the worker profile) in a second, public ledger. For example, the private ledger may include worker performance review, salary, etc., while the public ledger may include worker training information, such as date of training, title of training, professional or accredited endorsements associated with a training, etc. Companies may choose to store certain worker data, such as worker training data, to a public ledger accessible to other third parties. Thus, if worker 10A stops working for a first company and starts working for a second company, an authentication service 68I operated by the second company query the public ledger maintained by the consensus network to determine which trainings worker 10A completed prior to joining the second company. In some examples, sharing worker training data may enable worker 10A to start working for a second employer without re-taking trainings. In this way, companies may share certain worker data in a public ledger, which may increase efficiencies of workers and reducing time and costs that might otherwise be spent re-training workers when the worker has already completed the training.

Authentication service 68I may store raw data from PPE 30 or equipment 23 to the distributed ledger managed by the consensus network. In some examples, authentication service 68I receives raw data from the distributed ledger and analyzes the data to detect safety events. In some examples, authentication service 68I may receive raw data (e.g., from the distributed ledger, PPE 30, or equipment 23), analyze the data to detect safety events, and may store processed safety event data to the distributed ledger (e.g., via consensus network interface 65). For example, authentication service 68I may apply historical data and models 74B to data within distributed ledger to identify safety event signatures, anomalies or predicted occurrences of imminent safety events.

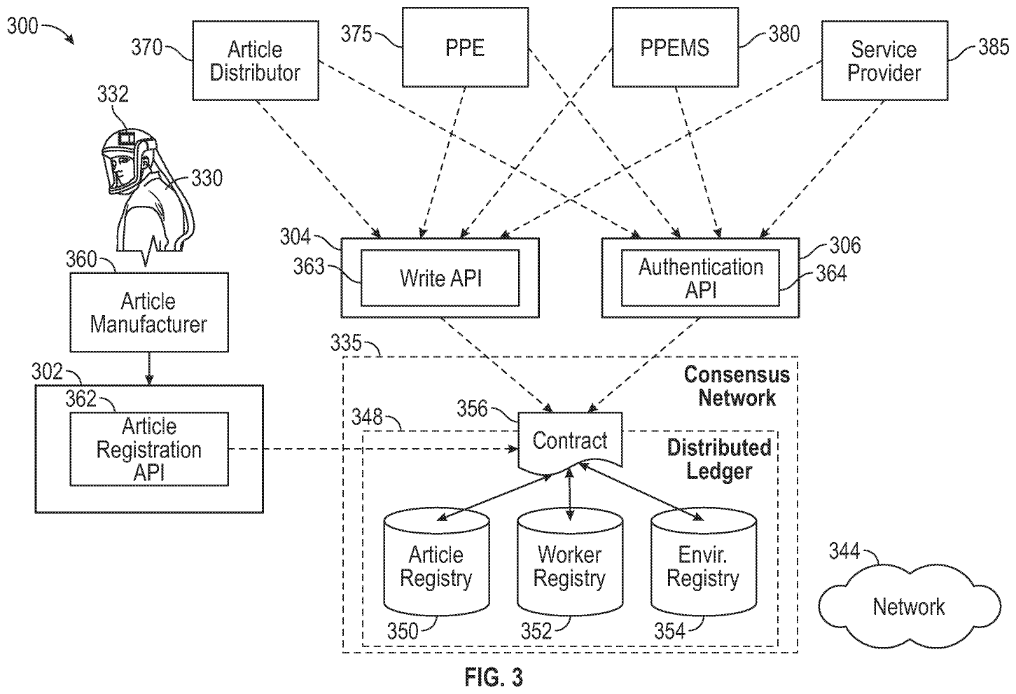

FIG. 3 is a block diagram depicting a system for authenticating an article of personal protective equipment, according to techniques described in this disclosure. System 300 in this example includes a consensus network 435 having a distributed ledger (e.g., blockchain) 438, as well as computing devices 302, 304, 306 that present respective article registration API 362, write properties API 363, and authentication API 364 for interacting with the consensus network 435 to register and authenticate transactions with the distributed ledger 438 using one or more smart contracts 356.

Article 430 may include an article of equipment (e.g., equipment 23 of FIG. 1) or an article of PPE (e.g., PPE 30 of FIG. 1). For example, article 430 may be a respirator, SRL, fitness tracker, among others. In some examples, article 430 includes a constituent component of an article of equipment 23 or PPE 30, such as a head top that constitutes part of a respirator 13.

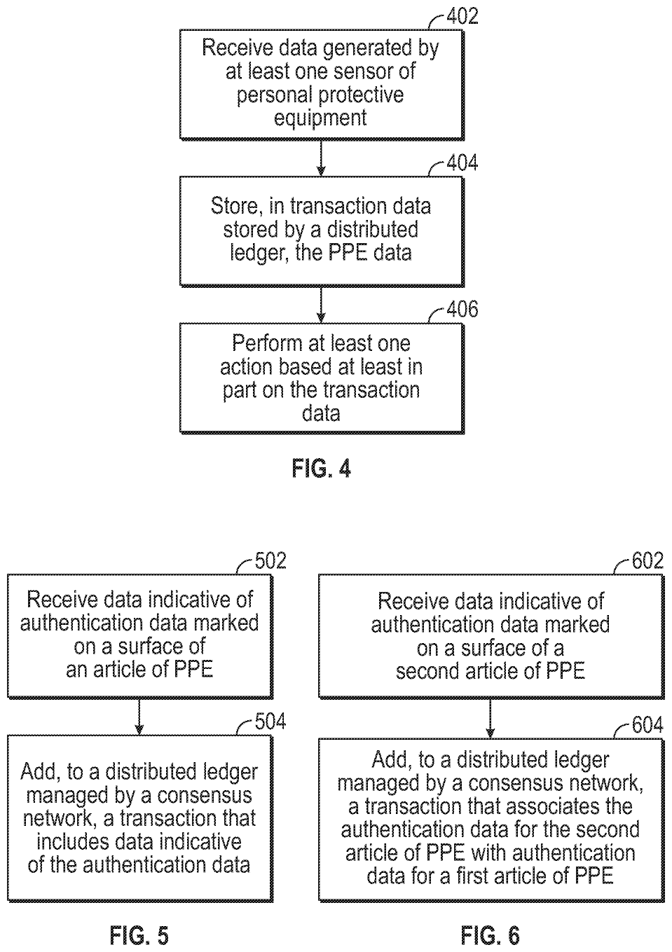

Consensus network 335 is a network of computing devices (or “nodes”) that implement a distributed ledger 338. Computing devices (not shown in FIG. 4) of the consensus network may represent any computing device able to execute smart contract 356. Consensus network 335 may, for instance, represent an Ethereum network of Ethereum virtual machines (EVMs), also known as an Ethereum blockchain platform, executing on hardware computing devices. Although only one smart contract 356 is illustrated, consensus network 335 may store and execute multiple different smart contracts 356 to facilitate the article registration and authentication techniques described herein.

Distributed ledger 338 is a shared transactional database that includes a plurality of blocks, each block (other than the root) referencing at least one block created at an earlier time, each block bundling one or more transactions registered with the distributed ledger 338, and each block cryptographically secured. Consensus network 335 receives transactions from transaction senders that invoke smart contract 356 to modify the distributed ledger 338. Consensus network 335 uses distributed ledger 338 for verification. Each block of distributed ledger 338 typically contains a hash pointer as a link to a previous block, a timestamp, and the transaction data for the transactions. By design, distributed ledgers are inherently resistant to modification of the transaction data. Functionally, distributed ledger 338 serves as a distributed ledger that can record transactions between parties efficiently and in a verifiable and permanent way.

Consensus network 335 may be a peer-to-peer network that manages distributed ledger by collectively adhering to a protocol for validating new blocks. Once recorded, the data in any given block of distributed ledger 338 cannot be altered retroactively without the alteration of all subsequent blocks and a collusion of a majority of the consensus network 335. Only one distributed ledger 338 is illustrated for simplicity, but multiple distributed ledgers 338 may be used with the described techniques. For example, consensus network 335 may manage one or more private ledgers and one or more public ledgers.

Contract 356 may represent a so-called “smart contract.” Contract 356 represents an executable script or program for performing a transaction for a party, or between parties, to modify state of distributed ledger 338. In examples of consensus network 335 that are Ethereum networks, contract 356 represents one or more autonomous scripts or one or more stateful decentralized applications that are stored in Ethereum distributed ledger 338 for later execution by the nodes of consensus network 335.

Contract 356 includes operations for modifying and viewing transaction data registered to distributed ledger. Such transaction data is categorized in FIG. 3 as article registry 350, worker registry 352, and environment registry 354. Distributed ledger 338 may store all data for article registry 350, worker registry 352, and environment registry 354 to a single distributed ledger address, or to multiple distributed ledger addresses, e.g., one address per registry/database. In the case of multiple distributed ledger addresses, contract 356 may represent multiple corresponding contracts.

System 300 includes a network 344 to transport data communications among computing devices of system 300. Network 344 may include the Internet. Communication links between network 344 and computing devices of system 300 are omitted from FIG. 3.

System 300 includes computing devices 302, 304, and 306. Each of computing devices 302, 304, and 306 presents a different application programming interface (API) for reading or modifying distributed ledger 338 using contract 356. Computing devices 302, 304, and 306 may communicate with consensus network 335 to request a new distributed ledger 338 transaction, to read transaction data, or to request consensus network 335 to perform another operation. Computing devices 302, 304, and 306 may send and receive data via network 344 to and from consensus network 335 using JavaScript Object Notation remote procedure call (JSON-RPC), a stateless light-weight remote procedure call. JSON-RPC may operate over sockets, over HyperText Transfer Protocol, or in other message passing environments. Computing devices 302, 304, and 306 may send and receive data for APIs 362, 363, and 364 via network 344. In some cases, a computing device may execute multiple of APIs 362, 363, and 364.

Computing device 302 presents article registration API 362, which presents methods for registering an article to distributed ledger 338. For example, article registration API 362 may include a register method configured to receive authentication data corresponding to article 330. In some examples, the authentication data includes a unique identifier corresponding to article 330.

An application (not shown) executed by computing device 302 may receive data invoking the register method of article registration API 362 and including data including the authentication data and, in some cases, other article data. The application of computing device 302 may send the authentication data to consensus network 335, at an address for contract 356, to invoke a register method of contract 356. The register method of contract 356 is configured to cause consensus network 335 to receive the authentication data corresponding to an article by adding at least one transaction to article registry 350 of distributed ledger 338. For example, article manufacturer(s) 360 may invoke the register method of article registration API 426 to register an article 330.

Article manufacturer 360 manufactures articles, e.g., article 330, where each article includes a code 441 representing respective authentication data ((e.g., printed on a surface of the article 330). For instance, article 330 may be an article of PPE (e.g., a hardhat, respirator, eyewear, etc.) or an article of equipment (e., HVAC equipment, a vehicle, construction equipment, etc.). In some examples, article 330 may be a constituent component (e.g., subcomponent) of an article of PPE or equipment. Article 330 may include a code 332, which may be an example of code 32 of FIG. 1. For example, code 332 may be printed, etched, affixed, or otherwise included on the article 330.

An operator, agent, or device controlled by article manufacturer 360 uses a computing device (e.g., computing device 16 of FIG. 1) to register each article 330 and the respective authentication data represented by the code 332 by invoking the register method of article registration API 362. Computing device 302, in turn, invokes the register method of contract 356 with the particular authentication data corresponding to article 330. Consensus network executes the register method to add a transaction to the distributed ledger 338 that modifies article registry 350 to add the authentication data to distributed ledger 338. For example, article registry 350 represents blocks of distributed ledger 338 that store authentication data corresponding to a respective article of a plurality of articles 330. In some examples, article registry 350 include a public key corresponding to the article manufacturer 360. For example, the register method of article registration API 362 may associate the authentication data for a particular article 330 with the public key for article manufacturer 360. In some examples, data registered to article registry 350 using article registration API 362 may include other descriptive data associated with a particular article 330, such as manufacturing date, lot number, manufacturer, type of article, article specifications, among others. Such data may be received and processed by the various methods, similarly to the authentication data for the article.

Computing device 304 presents write API 363 that includes at least one method for registering properties of article 330. For example, write API 363 may include an article properties method with which an entity may register properties of article 330. For example, the article properties method may enable an article distributor 370 to associate authentication data for a first article 330 with authentication data for a second article 330. For example, article distributor 370 may receive various articles 330 as subcomponents to a combined article, and assemble a combined article that includes the first article and second article. The article properties method may enable article distributor 370 to associate the first article and second article with one another and with the third, combined article.

In some examples, the article properties method may enable an entity to associate usage data with an article 330. For example, an article of PPE 375 may send usage data to consensus network 335 and associate the usage data with the article of PPE 375 within distributed ledger 348. In other words, the article properties method may permit article distributors 370, PPE 375, PPEMS 380, or service providers 385 to specify additional data about article 330. In such cases, the article properties method is configured to receive the additional data (e.g., usage data) associated with article 330 and invoke a corresponding method of contract 356 to cause consensus network 335 to register an association between authentication data (e.g., a unique identifier) corresponding to article 330 and additional data corresponding to article 330 in article registry 352 by adding at least one transaction to distributed ledger 338.

Write API 363 may include a worker properties method with which entities, also referred to as parties, (e.g., PPEMS 380) may register properties of a worker (e.g., worker 10A of FIG. 1). For example, the worker properties method may enable PPEMS 380 to register a worker 10A and specify data corresponding to worker 10A, such as biographical data (e.g., demographic data, work experience data, training data, etc.), safety event data for the worker, etc. An application executed by computing device 304 may respond to the invocation of the worker properties method of write API 363 by invoking contract 356 to cause consensus network 338 to register worker data for a particular worker. In some examples, invocation of the pathway method may cause consensus network 335 to register an association between an article 330 and worker 10A, such as data indicating that certain articles 330 are assigned to a worker 10A.

Write API 363 may include an environment properties method with which entities (e.g., PPEMS 380) may register properties of a worker (e.g., environment 8B of FIG. 1). For example, the environment properties method may enable PPEMS 380 to register an environment 8B and specify data corresponding to environment 8B, such as location, safety event data, hazards, etc. An application executed by computing device 304 may respond to the invocation of the environment properties method of write API 363 by invoking contract 356 to cause consensus network 338 to register environment data for a particular environment. In some examples, invocation of the environment properties method may cause consensus network 335 to register an association between an article 330 and an environment, such as data indicating that certain articles 330 are assigned to a particular environment 8B.

Computing device 306 presents authentication API 364 by which parties may request data from distributed ledger 338. In some examples, authentication API 364 includes an authentication method configured to receive authentication data for an article and return an indication of whether the consensus network 335 is able to authenticate the article.

Computing device 306 may receive data invoking the authentication method of authentication API 364 and including authentication data for an article for which authentication is being requested. Computing device 306 may send data including the authentication data for the article 330 to consensus network 335, at an address for contract 356, to invoke a corresponding authentication method of contract 356. The authentication method of contract 356 is configured to cause consensus network 335 to review transactions in distributed ledger 338 to determine whether the authentication data corresponding to article 330 exists within article registry 350 of distributed ledger 338. For example, the authentication method of contract 356 may compare the authentication data corresponding to article 330 to authentication data stored by article registry 350 to determine whether the authentication data corresponding to article 330 corresponds to (e.g., matches) authentication data within article registry 350. In some examples, the existence of the authentication data corresponding to article 330 within article registry 350 may indicate that article 330 is authentic. Responsive to determining that the authentication data corresponding to article 330 corresponds to authentication data within article registry 350, authentication API 364 may return an indication that the article 330 is authentic. However, in some examples, if the authentication method determines that the authentication data corresponding to article 330 does not correspond to authentication data within article registry 350, authentication API 364 may output an indication that the article 330 is not authentic (e.g., is counterfeit).

According to some scenarios, the authentication method may determine whether article 330 is authentic based on the received authentication information and a public key, private key pair. For example, the authentication method may determine that article 330 is authentic in response to determining that the received authentication information (e.g., a serial number received from PPEMS 380) corresponds to authentication information within the distributed ledger and that the public and private key pair is valid.

In some examples, the authentication method of authentication API 364 is configured to return an indication of whether the consensus network 335 is able to authenticate usage data associated with an article 330. For example, service provides 380 (e.g., insurance companies, inspection providers, maintenance providers, etc.) may cause computing device 306 to invoke the authentication method of contract 356 to cause consensus network 335 to compare PPE usage data received from service provider 385 with PPE usage data stored within article registry 350. In some example, authentication API 364 may output an indication that the PPE data is authentic in response to the authentication method determining that the received PPE usage data corresponds to (e.g., matches) the PPE usage data stored within article registry 350 (e.g., and in further response to determining the authentication data corresponding to article 330 matches authentication data within article registry 350). Similarly, authentication API 364 may output an indication that article 330 is not authentic in response to the authentication method determining that PPE usage data received from service provider 385 does not correspond the PPE usage data for article 330 as stored within article registry 350 (e.g., even if the authentication data corresponding article 330 matches authentication data within article registry 350).

In some examples, authentication API 364 includes a lookup method configured to output article registry data, worker registry data, or environment registry data to a requesting party. For example, service providers 385 (e.g., an insurance company) may request data regarding safety events at a particular environment. Authentication API 364 may retrieve safety event data from distributed ledger 348 and may output at least a portion of the safety event data to service provider 385. In some examples, service provider 385 may include an insurance company that may adjust insurance rates for a particular environment based on the safety event data. Because the safety event data is stored within distributed ledger 348, service provider 385 may be more confident that the data is correct (e.g., relative to other data storage techniques).

In some examples, one or more of article distributors 370, PPE 375, PPEMS 380, and/or service providers 385 may be a node within consensus network 335 and may host a copy of distributed ledger 338. For example, various article distributors (e.g., manufactures of final products) may store distributed ledger 338.

FIG. 4 is a flow diagram illustrating example operations of a computing device configured to authenticate an article of personal protective equipment, in accordance with one or more techniques of this disclosure. The techniques are described in terms of PPEMS 6 of FIGS. 1 and 2 and computing devices 304, 306 of FIG. 3. However, the techniques may be performed by other computing devices.

PPEMS 6 may receive PPE data generated by at least one sensor of an article of PPE (402). In some examples, the data generated by the at least one sensor includes PPE usage data. PPEMS 6 may receive the PPE usage data from an article of PPE (e.g., PPE 30 of FIGS. 1 and 2 or PPE 330 of FIG. 3) and authentication data for the article of PPE (e.g., data uniquely identifying the article of PPE).

PPEMS 6 may store the PPE usage data in transaction data of a distributed ledger managed by a consensus network (404). In some examples, PPEMS 6 may be a node on the consensus network and may store the PPE usage data in distributed ledger data store 74C. In another example, PPEMS 6 may send a command to computing device 304 to register the PPE usage data and the authentication data for the article of PPE, where the command includes the PPE usage data and the authentication data for the article of PPE. The command may cause computing device 306 may invoke an article properties method of Write API 363 to register the PPE usage data with authentication data for an article of PPE 330 as transaction data within the distributed ledger.

PPEMS 6 may perform at least one action based on the transaction data stored by the transaction data stored within the distributed ledger (406). For example, PPEMS 6 may query distributed ledger data store 74C to authentic an article of PPE, authentic PPE usage data, determine a change in a condition of an article of PPE over time, determine whether maintenance and/or inspections are being performed properly, among others. In some examples, PPEMS 6 performs the at least one action by at least sending a command to computing device 306, such as a command to authenticate an article of PPE, authenticate PPE usage data, determine a change in a condition of an article of PPE over time, determine whether maintenance and/or inspections are being performed properly, or a combination therein. The command may cause computing device 306 to invoke an authentication method of authentication API 364. In some examples, authentication API 364 may determine whether the article of PPE or PPE usage data is authentic. Computing device 306 may return an indication of whether the article of PPE or PPE usage data is authentic to PPEMS 6. Responsive to receiving the indication of whether the article of PPE or PPE usage data is authentic, PPEMS 6 may output a notification indicative of the determination.

As another example, PPEMS 6 may perform at least one action by determining whether inspections or maintenance of an article of PPE is being performed properly. For example, PPEMS 6 may send a command to computing device 306 to invoke the authentication method of authentication API 364 to determine whether PPE inspections and/or maintenance are performed according to a pre-determined schedule, performed by authorized service providers, or both. The authentication API 364 may return an indication of whether inspections and/or maintenance are being performed properly (e.g., according to schedule and by authorized service providers). PPEMS 6 may receive an indication of whether the maintenance and/or inspections are being performed properly. In response to receiving data that maintenance and/or inspections are not being performed properly, PPEMS 6 may output a notification. In some examples, the notification may include data indicating a reason why the maintenance and/or inspections are not being performed properly, possible remedies, or both.

FIG. 5 is flowchart illustrating an example mode of operation for a consensus network, according to techniques of this disclosure. The operations are described with respect to elements of system 300 of FIG. 3 but may be applied by systems having different architectures.

Distributed ledger 348 stores a smart contract 356 at a distributed ledger address. At least one node of the consensus network 345 receives data indicative of authentication data encoded on a code 332 marked on a surface on an article of PPE 330 (502). For example, the authentication data may include a unique identifier corresponding to article of PPE 330.

Consensus network 345 executes the register method of smart contract 356 to add a transaction to article registry 350 of distributed ledger 348 (504). The transaction includes data indicative of the authentication data such that the authentication data for article of PPE 330 is registered to the distributed ledger.

FIG. 6 is flowchart illustrating an example mode of operation for a consensus network, according to techniques of this disclosure. The operations are described with respect to elements of system 300 of FIG. 3 but may be applied by systems having different architectures.

Distributed ledger 348 stores a smart contract 356 at a distributed ledger address. In some examples, the distributed ledger 348 includes a transaction that includes authentication data indicative of a first article of PPE. At least one node of the consensus network 345 receives an indication of authentication data encoded in a code 332 marked on a surface of a second article of PPE 330 (602). For example, the authentication data may include a unique identifier corresponding to the second article 330.

Consensus network 345 executes the article properties method of smart contract 356 to add a transaction to article registry 450 of distributed ledger 348, where the transaction associates the authentication data for first article of PPE with the authentication data for the second article of PPE (604).

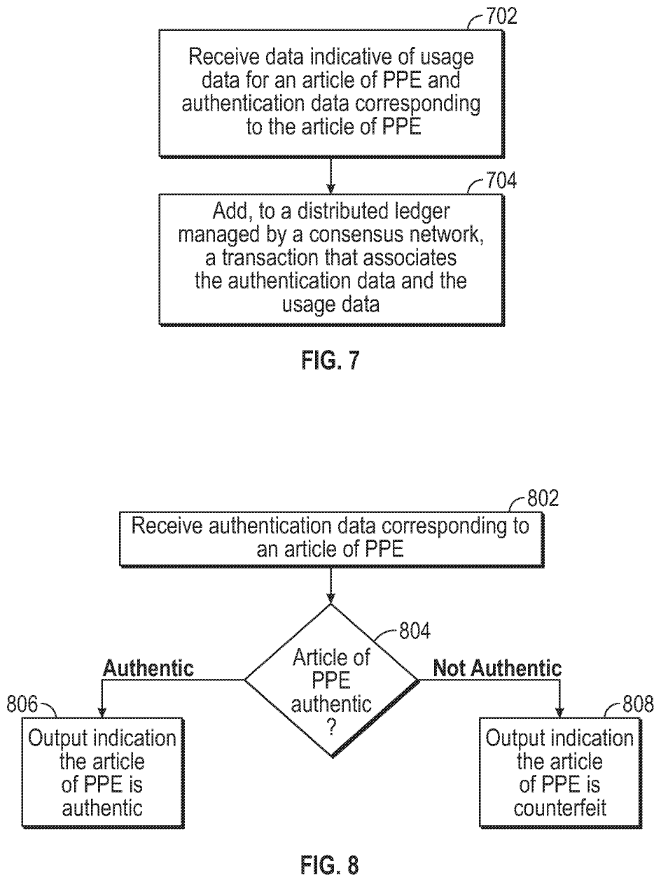

FIG. 7 is flowchart illustrating an example mode of operation for a consensus network, according to techniques of this disclosure. The operations are described with respect to elements of system 300 of FIG. 3 but may be applied by systems having different architectures.

Distributed ledger 348 stores a smart contract 356 at a distributed ledger address. A node of consensus network 345 receives authentication data corresponding to an article of PPE 330 and usage data corresponding to the article of PPE 330 (702).

Consensus network 345 executes the article properties method of smart contract 356 to add a transaction to article registry 350 of distributed ledger 348 that associates the authentication data for the article of PPE with the usage data for the article of PPE (704).

FIG. 8 is flowchart illustrating an example mode of operation for a consensus network, according to techniques of this disclosure. The operations are described with respect to elements of system 300 of FIG. 3 but may be applied by systems having different architectures.

Distributed ledger 348 stores a smart contract 356 at a distributed ledger address. A node of consensus network 345 receives a request for authentication of an article of PPE 330, the request specifying authentication data corresponding to the article of PPE (802). For example, the node of consensus network 345 may receive the request from a computing device, such as PPEMS 6 as illustrated in FIG. 1. In some examples, the request may include an indication of authentication data (e.g., a unique identifier) encoded in a code 332 on the article of PPE 330.

Consensus network 345 executes the authentication method of smart contract 356 to determine whether the article of PPE is authentic (804). A node of the consensus network may determine whether the article is authentic based at least in part on the authentication data corresponding to the article of PPE. For example, a node of the consensus network may determine that article 330 is authentic if a unique identifier corresponding to the article of PPE corresponds to (e.g., matches) an identifier stored within the distributed ledger managed by the consensus network, or may determine that the article is not authentic (e.g., counterfeit) if the unique identifier corresponding to the article of PPE does not correspond to an identifier stored within the distributed ledger.

Consensus network 345 outputs an indication of whether the article is authentic. For example, responsive to determining that the article is authentic (“Authentic” branch of 804), consensus network 345 outputs an indication that the article of PPE is authentic (806). For example, a node of consensus network 345 may send a notification to one or more of computing devices 60 of FIG. 1 indicating the article is authentic.

Responsive to determining that the article is not authentic (“Not Authentic” branch of 804), consensus network 345 outputs an indication that the article is not authentic (808). For example, a node of consensus network 345 may send a notification to one or more computing devices 60 of FIG. 1 indicating the article is not authentic.

Although the methods and systems of the present disclosure have been described with reference to specific exemplary embodiments, those of ordinary skill in the art will readily appreciate that changes and modifications may be made thereto without departing from the spirit and scope of the present disclosure.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

1. A system comprising:
an article of personal protective equipment (PPE) including at least one sensor configured to generate a stream of PPE data; and
at least one computing device configured to:
store, in transaction data stored by a distributed ledger managed by a consensus network of computing devices, the PPE data; and
perform, based at least in part on the transaction data stored by the distributed ledger, at least one action.

2. The system of claim 1, wherein the PPE data includes at least one of:
maintenance data for the article of PPE;
inspection data for the article of PPE;
a location of the article of PPE;
usage of the article of PPE; or
a physiological condition of a worker utilizing the article of PPE.

3. The system of claim 1,
wherein the at least one sensor includes a physiological sensor configured to generate physiological data indicative of one or more physiological characteristics of a worker.

4. The system of claim 1, further comprising:
an environmental sensor configured to generate environmental data indicative of one or more characteristics of a work environment,
wherein the at least one computing device is further configured to store the environmental data in the transaction data stored by the distributed ledger.

5. The system of claim 1, further comprising:
an article of equipment,
wherein the at least one computing device is further configured to store data indicative of the article of equipment in the transaction data stored by the distributed ledger.

6. The system of claim 1, wherein the computing device is further configured to:
receive an indication of a code embodied on the article of PPE, the code representative of authentication data for the article of PPE;
determine, based on the code and the transaction data stored by the distributed ledger, whether the article of PPE is authentic;
execute an action based on the determination.

7. The system of claim 1, wherein the PPE data is first PPE data, wherein the computing device is configured to perform the at least one action by being configured to:
receive second PPE data for the article of PPE;
determine, based on the first PPE data stored by the distributed ledger, whether the second PPE data is authentic; and
execute an action based on the determination.

8. The system of claim 1, wherein the PPE data is indicative of maintenance performed on the article of PPE, wherein the computing device is configured to perform the at least one action by being configured to:
determine, based on PPE data stored by the distributed ledger, whether maintenance of the article of PPE is being performed by an authorized entity or according to a pre-defined schedule; and execute an action based on the determination.

9. The system of claim 1, wherein the PPE data is indicative of inspections performed on the article of PPE, wherein the computing device is configured to perform the at least one action by being configured to:
determine, based on PPE data stored by the distributed ledger, whether inspections of the article of PPE are being performed by an authorized entity or according to a pre-defined schedule; and execute an action based on the determination.

10. The system of claim 1, wherein the distributed ledger is a first distributed ledger managed by a first type of consensus network, wherein the computing device is configured to perform the at least one action by being configured to:
receive second PPE data for the article of PPE from a second distributed ledger managed by a second type of consensus network;
determine whether the first PPE data is authentic based on the second PPE data; and
execute an action based on the determination.

11. The system of claim 1, wherein the computing device is configured to perform the at least one action by being configured to:
determine a condition of the article of PPE at a first time;
determine a condition of the article of PPE at a second, different time;
determine a difference in the condition of the article of PPE at the first time and the condition of the article of PPE at the second time; and
output an indication of the difference.

12. The system of claim 1, wherein the computing device is further configured to:
store, in the transaction data stored by the distributed ledger managed by the consensus network of computing devices, worker data, wherein the worker data includes worker training data;
determine, based on the worker data, whether a particular worker has participated in a particular training; and
output a notification based on the determination.

13. The system of claim 1, wherein the at least one computing device includes a memory device that stores at least a portion of the distributed ledger.

14. The system of claim 1, wherein the article of PPE includes the at least one computing device.

15. A computing device comprising:
at least one processor;
memory comprising instructions that, when executed, cause the at least one processor to:
receive a stream of PPE data from an article of personal protective equipment (PPE);
store, in transaction data stored by a distributed ledger managed by a consensus network of computing devices, the PPE data; and
perform, based at least in part on the transaction data stored by the distributed ledger, at least one action.

16. The computing device of claim 15, wherein the PPE data includes at least one of:
maintenance data for the article of PPE;
inspection data for the article of PPE;
a location of the article of PPE;
usage of the article of PPE; or
a physiological condition of a worker utilized the article of PPE.

17. The computing device of claim 15,
wherein the at least one sensor includes a physiological sensor configured to generate physiological data indicative of one or more physiological characteristics of a worker.

18. The computing device of claim 15, further comprising:
an environmental sensor configured to generate environmental data indicative of one or more characteristics of a work environment,
wherein the computing device is further configured to store the environmental data in the transaction data stored by the distributed ledger.

19. The computing device of claim 15, further comprising:
an article of equipment,
wherein the computing device is further configured to store data indicative of the article of equipment in the transaction data stored by the distributed ledger.

20. The computing device of claim 15, wherein the computing device is further configured to:
receive an indication of a code embodied on the article of PPE, the code representative of authentication data for the article of PPE;
determine, based on the code and the transaction data stored by the distributed ledger, whether the article of PPE is authentic;
execute an action based on the determination.

21-51. (canceled)
